(19) United States
(12) Patent Application Publication
(10) Pub. No.: US 2009/0203699 A1
(43) Pub. Date: Aug. 13, 2009

(54) AMINOMETHYLPYRIDINE DERIVATIVES, METHOD FOR PREPARING SAME AND THERAPEUTIC USE THEREOF

(75) Inventors: Francis Barth, Saint Georges D'Orques (FR); Christian Congy, Saint Gely Du Fesc (FR); Philippe Pointeau, Saint Clement De Riviere (FR); Murielle Rinaldi-Camona, Saint Georges D'Orques (FR); Lionel Barre, Montbazin (FR)

Correspondence Address: ANDREA Q. RYAN;SANOFI-AVENTIS U.S. LLC, 1041 ROUTE 202-206, MAIL CODE: D303A, BRIDGEWATER, NJ, 08807, US

(73) Assignee: Sanofi-Aventis

(21) Appl. No.: 12/249,140

(22) Filed: Oct. 10, 2008

Related U.S. Application Data
(63) Continuation of application No. PCT/FR2007/000620, filed on Apr. 12, 2007.

(30) Foreign Application Priority Data
Apr. 14, 2006 (FR) 06 03382

(51) Int. Cl. A61K 31/5377; C07D 401/12; A61K 31/4439; C07D 401/14; A61K 31/444; C07D 413/12; A61K 31/497

(52) U.S. Cl. 514/237.2; 546/276.4; 514/343; 546/194; 514/332; 544/124; 544/360; 514/255.05

(57) ABSTRACT

The present invention pertains to aminomethylpyridine derivatives, to their preparation and to their therapeutic application.

The present invention pertains to aminomethylpyridine derivatives, to their preparation and to their therapeutic application.

International patent application WO 03/082191 describes pyridine derivatives of formula:

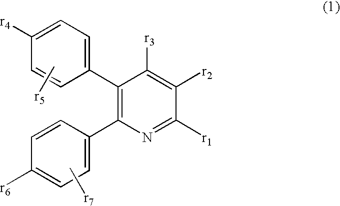

in which the substituents r₁ to r₇ have various values.

U.S. Pat. No. 5,916,905 describes pyridine derivatives of formula:

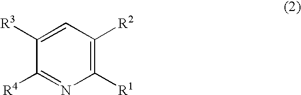

in which R³ and R⁴ may represent an aryl group and R² may represent an alkylcarbonylaminoalkyl group.

Patent application WO 2002/055502 describes compounds of formula:

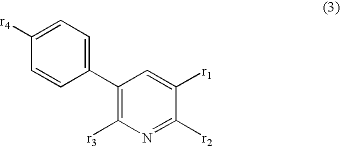

Patent application WO 2006/113704 describes compounds of formula:

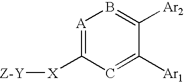

in which B may represent a nitrogen atom, while A and C represent carbon atoms.

Patent application WO 2004/111034 describes pyrazine derivatives of formula:

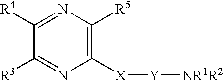

These compounds are described as being CB₁ receptor modulators.

Patent application WO 2006/042955 describes pyridine derivatives which are cannabinoid CB₁ receptor antagonists, of formula:

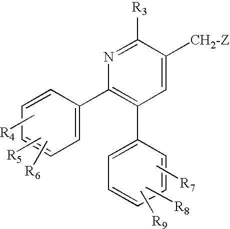

New aminomethylpyridine derivatives have now been found which possess peripherally and/or centrally located cannabinoid CB₁ receptor antagonist properties.

The present invention provides compounds conforming to the formula:

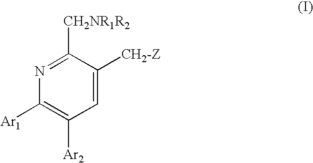

in which:
Z represents a group N(R₃) XR₄, N(R₃)COOR₅ or OCON(R₃)R₅;
X represents a group —CO—, —SO₂—, —CON(R₆)— or —CSN(R₅)—;
R₁ and R₂ represent, each independently of one another, a hydrogen atom or a (C₁-C₇)alkyl, or R₁ and R₂, together with the nitrogen atom to which the are bonded, form a saturated or unsaturated 3- to 8-membered heterocyclic radical which may contain one or more other heteroatoms selected from an oxygen, sulphur or nitrogen atom, said radical being unsubstituted or substituted by one or more (C₁-C₄)alkyl groups;
R₁ represents a hydrogen atom or a (C₁-C₄)alkyl group;
R₄ represents;
a (C₃-C₁₀)alkyl group unsubstituted or substituted by a CF₃ group;
a nonaromatic (C₃-C₁₂) carbocyclic radical unsubstituted or substituted one or more times by identical or different substituents selected from a (C₁-C₄)alkyl, hydroxyl, (C₁-C₄)alkoxy, (C₁-C₄)alkylthio and cyano group;
a heterocyclic radical of 3 to 8 atoms which contains oxygen, sulphur or nitrogen, is saturated or unsaturated and is unsubstituted or substituted by one or more identical or different substituents selected from a halogen atom and a (C₁-C₄)alkyl, hydroxyl, trifluoromethyl, (C₁-C₄)alkoxy, trifluoromethoxy, trifluoromethylthio, (C₁-C₄)alkylthio, cyano or nitro group or by an oxo group;
an indolyl which is unsubstituted or substituted by a halogen atom or by a (C₁-C₄)alkyl, trifluoromethyl, hydroxyl, (C₁-C₄)alkoxy, trifluoromethoxy, trifluoromethyl thio, (C₁-C₄)alkylthio, cyano or nitro group;
a tetrahydronaphthyl; a naphthyl;
a benzothiophenyl or a benzofuryl;
a phenyl which is unsubstituted or substituted one or more times by identical or different substituents selected from a halogen atom, a (C₁-C₄)alkyl, trifluoromethyl, trifluoromethoxy, hydroxyl, (C₁-C₄)alkoxy, (C₁-C₄)alkylthio, trifluoromethylthio, cyano, nitro, (C₁-C₄)alkanoyl or phenyl group or a group S(O)ₙAlk, OS(O)ₙAlk or NR₇R₈;
a benzodioxyl;
a phenoxymethylene or a 1-phenoxyethylene, the phenyl groups being unsubstituted or substituted one or more times by identical or different substituents selected from a halogen atom, a (C₁-C₄)alkyl, trifluoromethyl, trifluoromethoxy, hydroxyl, (C₁-C₄)alkoxy, (C₁-C₄)alkylthio, trifluoromethylthio, cyano, nitro, (C₁-C₆)alkanoyl or phenyl group or a group S(O)ₙAlk, OS(O)ₙAlk or NR₇R₈; the methylene or ethylene groups being unsubstituted or substituted one or more times by a (C₁-C₄)alkyl group or by a (C₃-C₇)cycloalkyl;
a phenylcyclopropyl, the phenyl group being unsubstituted or substituted one or more times by identical or different substituents selected from a halogen atom, a (C₁-C₄)alkyl, trifluoromethyl, trifluoromethoxy, hydroxyl, (C₁-C₄)alkoxy, (C₁-C₄)-alkylthio, trifluoromethylthio, cyano, nitro, (C₁-C₄)alkanoyl or phenyl group or a group S(O)ₙAlk, OS(O)ₙAlk or NR₇R₈:
a (C₁-C₂)alkylene substituted by one or two identical or different substituents selected from:
(i) a nonaromatic C₃-C₁₂ carbocyclic radical which is unsubstituted or substituted one or more times by a (C₁-C₄)alkyl group;
(ii) a phenyl which is unsubstituted or substituted by one or more identical or different substituents selected from a halogen atom, a (C₁-C₄)alkyl, hydroxyl, trifluoromethyl, (C₁-C₄)alkoxy, (C₁-C₄)alkyl thio, trifluoromethoxy, trifluoromethylthio, (C₁-C₄)alkanoyl, cyano, nitro or phenyl group or a group S(O)ₙAlk, OS(O)ₙAlk or NR₇R₈;
(iii) a heterocyclic radical of 3 to 8 atoms which contains oxygen, sulphur or nitrogen, is saturated or unsaturated and is unsubstituted or substituted by one or more identical or different, substituents selected from a halogen atom and a (C₁-C₄)alkyl, hydroxyl, trifluoromethyl, (C₁-C₄)alkoxy, trifluoromethoxy, trifluoromethylthio, (C₁-C₄)alkyl thio, cyano or nitro group;
additionally, when X represents a group —CON(R₆)— or —CSN(R₆)—, R₄ may represent a (C₁-C₄)alkanoyl group or a benzoyl or benzylcarbonyl group, the phenyl group of said groups being unsubstituted or substituted by identical or different substituents selected from a halogen atom, a (C₁-C₄)alkyl, trifluoromethyl, trifluoromethoxy, hydroxyl, (C₁-C₄)alkoxy, (C₁-C₄)alkylthio, trifluoromethylthio, cyano, nitro, (C₁-C₄)alkanoyl or phenyl group or a group S(O)ₙAlk, OS(O)ₙAlk or NR₇R₈;
R₅ represents a phenyl which is unsubstituted or substituted one or more times by identical or different substituents selected from a halogen atom, a (C₁-C₄)alkyl, trifluoromethyl, trifluoromethoxy, cyano, nitro, (C₁-C₄)alkoxy, (C₁-C₄)alkylthio or trifluoromethylthio group or a group S(O)ₙAlk, OS(O)ₙAlk or NR₇R₈;
R₆ represents a hydrogen atom or a (C₁-C₄)alkyl group;
or R₄ and R₆, together with the nitrogen atom to which they are bonded, form a heterocyclic radical of 3 to 8 atoms which does or does not contain a second heteroatom selected from an oxygen, sulphur or nitrogen atom, unsubstituted or substituted one or more times by a (C₁-C₄)alkyl group; a (C₁-C₄)alkanoyl group; a group NR₇R₈ or CONR₇R₈; a phenyl group which is unsubstituted or substituted one or more times by a halogen atom, a (C₁-C₄)alkyl, (C₁-C₄)alkoxy or trifluoromethyl, (C₁-C₄)alkylthio, trifluoromethoxy or trifluoromethylthio group or a group OS(O)ₙAlk, S(O)ₙAlk or NR₇R₈;
R₇ and R₈ represent, each independently of one another, a hydrogen atom or a (C₁-C₄)alkyl group or R₇, and R₈, together with the nitrogen atom to which they are bonded, form a saturated heterocyclic radical of 4 to 8 atoms which may contain another heteroatom selected from a nitrogen, oxygen or sulphur atom;
Ar₁ and Ar₂ represent, each independently of one another, a phenyl which is unsubstituted or substituted by a halogen atom, a (C₁-C₆)alkyl, (C₁-C₆)alkoxy, (C₁-C₆)alkylthio, trifluoromethyl, trifluoromethoxy, trifluoromethylthio, cyano or nitro group or a group S(O)ₙAlk, OS(O)ₙAlk or NR₇R₈;
n represents 0, 1 or 2;
Alk represents a (C₁-C₇)alkyl group;
   in the form of a base or addition salt, or in the form of a hydrate or solvate.

The present invention more particularly provides the compounds of formula (I) in which:
Z represents a group N(R₃)XR₄, N(R₃)COOR₅ or OCON(R₃)R₅;
X represents a group —CO—, —SO₂—, —CON(R₆)— or —CSN(R₆)—;
R₁ and R₂ represent, each independently of one another, a hydrogen atom or a (C₁-C₇)alkyl, or R₁ and R₂, together with the nitrogen atom to which they are bonded, form a saturated or unsaturated 3- to 8-membered heterocyclic radical which may contain one or more other heteroatoms selected from an oxygen, sulphur or nitrogen atom, said radical being unsubstituted or substituted by one or more (C₁-C₄)alkyl groups;
R₃ represents a hydrogen atom or a (C₁-C₄)alkyl group;
R₄ represents:
a (C₃-C₁₀)alkyl group unsubstituted or substituted, by a CF₃ group;
a nonaromatic (C₃-C₁₂) carbocyclic radical unsubstituted or substituted one or more times by identical or different substituents selected from a (C₁-C₄)alkyl, hydroxyl, (C₁-C₄)alkoxy, (C₁-C₄)alkylthio and cyano group;
a heterocyclic radical of 4 to 8 atoms which contains oxygen, sulphur or nitrogen, is saturated or unsaturated and is unsubstituted or substituted by one or more identical or different substituents selected from a halogen atom and a (C₁-C₄)alkyl, hydroxyl, trifluoromethyl, (C₁-C₄)alkoxy, trifluoromethoxy, (C₁-C₄)alkylthio, cyano or nitro group;
an indolyl which is unsubstituted or substituted by a halogen atom or by a (C₁-C₄)alkyl, trifluoromethyl, hydroxyl, (C₁-C₄)alkoxy, trifluoromethoxy, (C₁-C₄)alkylthio, cyano or nitro group;
a tetrahydronaphth-1-yl or -2-yl; a naphth-1-yl or -2-yl;
a benzothiophenyl or a benzofuryl;
a phenyl which is unsubstituted or substituted one or more times by identical or different substituents selected from a halogen atom, a (C₁-C₄)alkyl, trifluoromethyl, trifluoromethoxy, hydroxyl, (C₁-C₄)alkoxy, (C₁-C₄)alkylthio, trifluoromethylthio, cyano, nitro, (C₁-C₄)alkanoyl or phenyl group or a group S(O)ₙAlk, OS(O)ₙAlk or NR₇R₈;
a benzodioxyl;
a phenoxymethylene or a 1-phenoxyethylene, the phenyl groups being unsubstituted or substituted one or more times by identical or different substituents selected from a halogen atom, a (C₁-C₄)alkyl, trifluoromethyl, trifluoromethoxy, hydroxyl, (C₁-C₄)alkoxy, (C₁-C₄)alkylthio, trifluoromethylthio, cyano, nitro, (C₁-C₄)alkanoyl or phenyl group or a group S(O)ₙAlk, OS(O)ₙAlk or NR₇R₈; the methylene or ethylene groups being unsubstituted or substituted one or more times by a (C₁-C₄)alkyl group or by a (C₃-C₇)cycloalkyl;
a phenyl cyclopropyl, the phenyl group being unsubstituted or substituted one or more times by identical or different substituents selected from a halogen atom, a (C₁-C₄)alkyl, trifluoromethyl, trifluoromethoxy, hydroxyl, (C₁-C₄)alkoxy, (C₁-C₄)-alkylthio, trifluoromethylthio, cyano, nitro, (C₁-C₄)alkanoyl or phenyl group or a group S(O)ₙAlk, OS(O)ₙAlk or NR₇R₈;
a (C₁-C₂)alkylene substituted by one or two identical or different substitutents selected from:
(i) a nonaromatic C₃-C₁₂ carbocyclic radical which is unsubstituted or substituted one or more times by a (C₁-C₄)alkyl group;
(ii) a phenyl which is unsubstituted or substituted by one or more identical or different substituents selected from a halogen atom, a (C₁-C₄)alkyl, hydroxyl, trifluoromethyl, (C₁-C₄)alkoxy, (C₁-C₄)alkyl thio, trifluoromethoxy, trifluoromethylthio (C₁-C₄)alkanoyl, cyano, nitro or phenyl group or a group S(O)ₙAlk, OS(O)ₙAlk or NR₇R₈;
(iii) a heterocyclic radical of 4 to 8 atoms which contains oxygen, sulphur or nitrogen, is saturated or unsaturated and is unsubstituted or substituted by one or more identical or different substituents selected from a halogen atom and a (C₁-C₄)alkyl, hydroxyl, trifluoromethyl, (C₁-C₄)alkoxy, trifluoromethoxy, (C₁-C₄)alkylthio, cyano or nitro group;
additionally, when X represents a group —CON(R₆)— or —CSN(R₆)—, R₄ may represent a (C₁-C₆)alkanoyl group or a benzoyl or benzylcarbonyl group, the phenyl group of said groups being unsubstituted or substituted by identical or different substituents selected from a halogen atom, a (C₁-C₄)alkyl, trifluoromethyl, trifluoromethoxy, hydroxyl, (C₁-C₄)alkoxy, (C₁-C₄)alkylthio, trifluoromethylthio, cyano, nitro, (C₁-C₄)alkanoyl or phenyl group or a group S(O)ₙAlk, OS(O)ₙAlk or NR₇R₈;
R₅ represents a phenyl which is unsubstituted or substituted one or more times by identical or different substituents selected from a halogen atom or a (C₁-C₄)alkyl, trifluoromethyl, trifluoromethoxy, cyano, nitro, (C₁-C₄)alkoxy, (C₁-C₄)alkylthio, trifluoromethylthio, S(O)ₙAlk or OS(O)ₙAlk group;
R₆ represents a hydrogen atom or a (C₁-C₄)alkyl group;
or R₄ and R₆, together with the nitrogen atom to which they are bonded, form a heterocyclic radical of 4 to 8 atoms which does or does not contain a second heteroatom selected from an oxygen, sulphur or nitrogen atom, unsubstituted or substituted one or more times by a (C₁-C₄)alkyl group; a (C₁-C₄)-alkanoyl group; a group NR₇R₈ or CONR₇R₈; a phenyl group which is unsubstituted or substituted one or more times by a halogen atom, a (C₁-C₄)alkyl, (C₁-C₄)alkoxy or trifluoromethyl, (C₁-C₄)alkyl thio, trifluoromethoxy or trifluoromethylthio group or a group OS(O)ₙAlk or S(O)ₙAlk group;
R₇ and R₈ represent, each independently of one another, a hydrogen atom or a (C₁-C₄)alkyl group or R₇ and R₈, together with the nitrogen atom to which they are bonded, form a saturated heterocyclic radical of 4 to 8 atoms which may contain another heteroatom selected from a nitrogen, oxygen or sulphur atom;
Ar₁ and Ar₂ represent, each independently of one another, a phenyl which is unsubstituted or substituted by a halogen atom, a (C₁-C₆)alkyl, (C₁-C₆)alkoxy, trifluoromethyl, trifluoromethoxy, trifluoromethylthio, cyano or nitro group or a group S(O)ₙAlk or OS(O)ₙAlk group;
n represents 0, 1 or 2;
Alk represents a (C₁-C₄)alkyl group;
   in the form of a base or addition salt, or in the form of a hydrate or solvate.

Singled out among the compounds of formula (I) provided by the invention are:
the compounds of formula (IA) in which Z represents a group —N(R₃)COR₄ and R₁ to R₄, Ar₁ and Ar₂ are as defined for (I);
the compounds of formula (IB) in which Z represents a group —N(R₃)SO₂R₄ and R₁ to R₄, Ar₁ and Ar₂ are as defined for (I);
the compounds of formula (IC) in which Z represents a group —N(R₃)CON(R₆)R₄ and R₁ to R₄, Ar₁ and Ar₂ are as defined for (I);
the compounds of formula (ID) in which Z represents a group —N(R₃)CSN(R₆)R₄ and R₁ to R₄, Ar₁ and Ar₂ are as defined for (I);
the compounds of formula (IE) in which Z represents a group —N(R₃)COOR₅ and R₁ to R₄, Ar₁ and Ar₂ are as defined for (I);
the compounds of formula (IF) in which Z represents a group —O—CO—NR₃R₅ and R₁ to R₄, Ar₁ and Ar₂ are as defined for (I).

More particularly the present invention provides the compounds of formula:

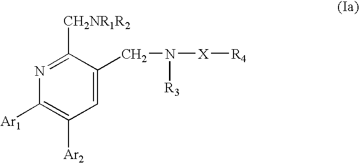

in which:
X represents a group —CO—, —SO₂— or —CON(R₆)—;
R₁ and R₂ are as defined for (I);
R₃ represents a hydrogen atom or a (C₁-C₄)alkyl group;
R₄ represents;
a (C₃-C₁₀)alkyl group;
a nonaromatic (C₃-C₁₂) carbocyclic radical unsubstituted or substituted one or more times by
a (C₁-C₄)alkyl group;
a heterocyclic radical of 4 to 8 atoms which contains oxygen, sulphur or nitrogen, is saturated or unsaturated and is unsubstituted or substituted by one or more identical or different substituents selected from a halogen atom and a (C₁-C₄)alkyl, hydroxyl, trifluoromethyl, (C₁-C₄)alkoxy, trifluoromethoxy, (C₁-C₄)alkylthio, cyano or nitro group;
an indolyl unsubstituted or substituted on the nitrogen atom by a halogen atom, a (C₁-C₄)alkyl group or a (C₁-C₄)alkoxy group;
a phenyl which is unsubstituted or substituted one or more times by identical or different substituents selected from a halogen atom, a (C₁-C₄)alkyl, trifluoromethyl, trifluoromethoxy, (C₁-C₄)alkoxy, (C₁-C₄)alkylthio, trifluoromethylthio, cyano, (C₁-C₄)alkanoyl or phenyl group or a group S(O)ₙAlk or OS(O)ₙAlk;
a benzyl which is unsubstituted or substituted one or more times by identical or different substituents selected from a halogen atom, a (C₁-C₄)alkyl, trifluoromethyl, trifluoromethoxy, trifluoromethylthio, (C₁-C₄)alkoxy, cyano or phenyl group or a group S(O)ₙAlk or OS(O)ₙAlk;
R₆ represents a hydrogen atom or a (C₁-C₄)alkyl group;
or R₄ and R₆, together with the nitrogen atom to which they are bonded, form a heterocyclic radical of 4 to 8 atoms which does or does not contain a second heteroatom selected from an oxygen, sulphur or nitrogen atom, unsubstituted or substituted one or more times by a (C₁-C₄)alkyl group; a (C₁-C₄)alkanoyl group; a group NR₇R₈ or CONR₇R₈; a phenyl group unsubstituted or substituted one or more times by a halogen atom or a (C₁-C₄)alkyl, (C₁-C₄)alkoxy or trifluoromethyl group;
R₇ and R₈ represent, each independently of one another, a hydrogen atom or a (C₁-C₄)alkyl group, or R₇ and R₈, together with the nitrogen atom to which they are bonded, form a heterocyclic radical selected from piperidinyl, pyrrolidinyl, piperazinyl, N-methylpiperazinyl, azepinyl or morpholinyl;
Ar₁ and Ar₂ represent, each independently of one another, a phenyl group which is unsubstituted or substituted by a halogen atom, a (C₁-C₆)alkyl (C₁-C₆)alkoxy, trifluoromethyl, trifluoromethylthio or trifluoromethoxy group or a group S(O)ₙAlk or OS(O)ₙAlk;
n represents 0, 1 or 2;
Alk represents a (C₁-C₄)alkyl group;
   in the form of a base or addition salt, or in the form of a hydrate oz solvate.

More particularly still, the present invention provides the compounds of formula:

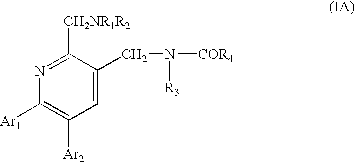

in which:
R₁ and R₂ represent, each independently of one another, a (C₁-C₇)alkyl, or R₁ and R₂, together with the nitrogen atom to which they are bonded, form a radical selected from azeridinyl, azetidinyl, pyrrolidinyl, piperidinyl, azepinyl, piperazinyl, N-methylpiperazinyl, morpholinyl, imidazolyl, pyrazolyl, tetrazolyl and triazolyl;
R₃ represents a hydrogen atom or a methyl;
R₄ represents:
a (C₅-C₁₀)alkyl;
a (C₅-C₇)cycloalkyl unsubstituted or substituted one or more times by a methyl;
a heterocyclic radical of 4 to 8 atoms which contains oxygen, sulphur or nitrogen, is saturated and is unsubstituted or substituted one or more times by a methyl;
a phenyl substituted one or more times by a halogen atom groups selected independently from or a trifluoromethyl, trifluoromethoxy, trifluoromethylthio, (C₁-C₄)alkoxy, (C₁-C₄)alkylthio, SO₂Alk or OSO₂Alk group;
Ar₁ and Ar₂ represent, each independently of one another, a phenyl substituted by one or two substituents selected independently from a halogen atom and a methoxy, methylthio, trifluoromethylthio, trifluoromethoxy, SO₂Alk or OSO₂Alk group;
   in the form of a base or addition salt, or in the form of a hydrate or solvate.

More particularly still, the present invention provides the compounds of formula:

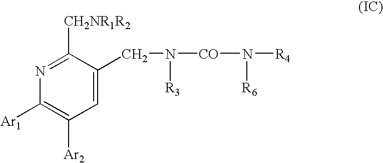

in which:
R₁ and R₂ represent, each independently of one another, a (C₁-C₇)alkyl, or R₁ and R₂, together with the nitrogen atom to which they are bonded form a radical selected from azeridinyl, azetidinyl, pyrrolidinyl, piperidinyl, azepinyl, piperazinyl, N-methylpiperazinyl, morpholinyl, imidazolyl, pyrazolyl, tetrazolyl and triazolyl;
R₃ represents a hydrogen atom or a methyl;
R₄ represents:
a (C₅-C₁₀)alkyl;
a (C₅-C₇)cycloalkyl unsubstituted or substituted one or more times by a methyl;
a heterocyclic radical of 4 to 8 atoms which contains oxygen, sulphur or nitrogen, is saturated and is unsubstituted or substituted one or more times by a methyl;
a phenyl substituted one or more times by groups selected independently from a halogen atom and a trifluoromethyl, trifluoromethoxy, trifluoromethylthio, (C₁-C₄)alkoxy, (C₁-C₄)alkylthio, SO₂Alk or OSO₂ Alk group;
R₆ represents a hydrogen atom or a methyl;
Ar₁ and Ar₂ represent, each independently of one another, a phenyl substituted by one or two substituents selected independently from a halogen atom and a methoxy, methylthio, trifluoromethylthio, trifluoromethoxy, SO₂Alk or OSO₂Alk group;
   in the form of a base or addition salt, or in the form of a hydrate or solvate.

The compounds of formula (I) may contain one or more asymmetric carbon atoms. They may therefore exist in the form of enantiomers or diastereoisomers. These enantiomers, diastereoisomers, and mixtures thereof, including the racemic mixtures, form part of the invention.

The compounds of formula (I) may exist in the form of bases or addition salts with acids. Such addition salts form part of the invention.

These salts may be prepared with pharmaceutically acceptable acids, although the salts of other acids, which are useful, for example, for the purification or isolation of compounds of formula (I), likewise form part of the invention.

The compounds of formula (I) may likewise exist in the form of hydrates or solvates, in other words in the form of associations or combinations with one or more molecules of water or with a solvent. Such hydrates and solvates likewise form part of the invention.

In the context of the present invention the following terms have the following definitions:
a halogen atom: a fluorine, a chlorine, a bromine or an iodine;
a (C₁-C₄)alkyl or respectively (C₁-C₆)alkyl, (C₁-C₇)alkyl, (C₃-C₁₀)alkyl or (C₅-C₁₀)alkyl group: a (C₁-C₄), or respectively (C₁-C₆), (C₁-C₇), (C₃-C₁₀) or (C₅-C₁₀), linear or branched, saturated aliphatic group. Examples include the groups methyl, ethyl, propyl, isopropyl, butyl, isobutyl, tert-butyl, pentyl, hexyl, 1-ethylpropyl, 1-propylbutyl, 2-propylpentyl, 5-methylnonyl, 4-methylheptyl, 4-methyl-2,6-dimethylheptyl, etc.;
a (C₁-C₄)alkoxy or respectively (C₁-C₆)alkoxy group: an O-alkyl radical in which the alkyl group is as defined above.

The nonaromatic C₃-C₁₂ carbocyclic radicals comprise bridged or fused monocyclic or polycyclic radicals. The monocyclic radicals include the cycloalkyls, for example cyclopropyl, cyclobutyl, cyclopentyl, cyclohexyl, cycloheptyl and cyclooctyl, with cyclohexyl and cyclopentyl being preferred. The fused, bridged or spiro dicyclic or tricyclic radicals include, for example, the radicals norbornyl, bornyl, isobornyl, noradamantyl, adamantyl, spiro[5.5]undecanyl, bicyclo-[2.2.1]heptyl, bicyclo[3.2.1]octyl, and bicyclo-[3.1.1]heptyl.

The 3- to 8-membered, nitrogen-containing heterocyclic radicals formed by two substituents together with the nitrogen atom to which they are bonded comprise saturated radicals such as azeridinyl, azetidinyl, pyrrolidinyl, piperidyl, perhydroazepinyl and perhydroazocinyl; and saturated or unsaturated radicals additionally containing a second heteroatom selected from an oxygen, sulphur or nitrogen atom, such as imidazolidinyl, pyrazolidinyl, piperazinyl, morpholinyl, thiomorpholinyl, imidazolyl, pyrazolyl, isothiazolyl, isoxazolyl, triazolyl, tetrazolyl, oxazolyl and thiazolyl. The 3- to 8-membered, unsaturated, nitrogen-containing heterocyclic radicals further comprising one or more heteroatoms comprise imidazolyl, pyrrolyl, pyrazolyl, isothiazolyl and isoxazolyl.

The heterocyclic radicals of 3 to 8 atoms which contain oxygen, sulphur or nitrogen and are saturated or unsaturated comprise, in particular, furyl, tetrahydrofuryl, thienyl and pyrrolyl.

Singled out more particularly are the compounds of formula (IA) in which:
Z represents a group NHCOR₄;
R₁ and R₂ represent, each independently of one another, a (C₁-C₇)alkyl, or R₁ and R₂, together with the nitrogen atom to which they are bonded, form a radical selected from azeridinyl, azetidinyl, pyrrolidinyl, piperidinyl, azepinyl, piperazinyl, N-methylpiperazinyl, morpholinyl, imidazolyl, pyrazolyl, tetrazolyl and triazolyl;
R₄ represents a 2-propylpentyl, 1-propylbutyl, 5-methylnonyl, 4-methylheptyl or 4-methyl-2,6-dimethylheptyl group, a cyclopentyl, tetramethyl-cyclopentyl, cyclohexyl, tetrahydrofuranyl, pyrrolidinyl, 1,1,4,4-tetramethylcyclopentyl, 2,2,5,5-tetramethylfuranyl or 2,2,5,5-tetramethyl-pyrrolidinyl group, or a phenyl group which is unsubstituted or substituted by a halogen atom, a trifluoromethyl, a trifluoromethoxy or a trifluoromethyl thio or by a group SO₂Alk or OSO₂Alk;
and/or Ar₁ and Ar₂ represent, each independently of one another, a phenyl substituted one or more times by substituents selected independently from a chlorine or bromine atom or a methoxy or methylthio group;
   in the form of a base or addition salt, or in the form of a hydrate or solvate.

Likewise singled out are the compounds of formula (IC) in which:
Z represents a group —NHCONHR₄;
R₁ and R₂ represent, each independently of one another, a (C₁-C₇)alkyl, or R₁, and R₂, together with the nitrogen atom to which they are bonded, form a radical selected from azeridinyl, azetidinyl, pyrrolidinyl piperidinyl, azepinyl, piperazinyl, N-methylpiperazinyl, morpholinyl, imidazolyl, pyrazolyl, tetrazolyl and triazolyl;
R₄ represents a cyclohexyl group or a phenyl group which is unsubstituted or substituted by a halogen atom or by a methoxy, trifluoromethyl, trifluoromethoxy or trifluoromethyl-thio group;
Ar₁ and Ar₂ represent, each independently of one another, a phenyl unsubstituted or substituted one or more times by substituents selected independently from a chlorine or bromine atom or a methoxy or methylthio group;
   in the form of a base or addition salt, or in the form of a hydrate or solvate.

The compounds of the invention that have been described include in particular the following compounds:
N-{[6-(4-chlorophenyl)-5-(2,4-dichlorophenyl)-2-(pyrrolidin-ylmethyl)pyridin-3-yl]methyl}-4-(trifluoromethoxy)benzamide;
N-{[6-(4-bromophenyl)-5-(2,4-dichlorophenyl) 2-(tetrazol-2-ylmethyl)pyridin-3-yl]methyl}-4-(trifluoromethyl)benzamide;
N-{[6-(4-bromophenyl)-5-(2,4-dichlorophenyl)-2-[1,2,4]triazol-1-ylmethylpyridin-3-yl]methyl}-4-(trifluoromethyl)benzamide;
N-{[6-(4-chlorophenyl)-5-(2,4-dichlorophenyl)-2-(pyrrolidin-1-ylmethyl) pyridin-3-yl]methyl}-2-propyl-pentanamide;
N-{[6-(4-chlorophenyl)-5-(2,4-dichlorophenyl)-2-(pyrrolidin-1-ylmethyl)pyridin-3-yl]methyl}-4-((trifluoromethyl)thio)benzamide;
N-{[6-(4-chlorophenyl)-5-(2,4-dichlorophenyl)-2-(piperidin-1-yl]methyl)pyridin-3-yl]methyl}-4-(trifluoromethoxy)benzamide;
N-{[6-(4-chlorophenyl)-5-(2,4-dichlorophenyl)-2-(morpholin-4-yl]methyl)pyridin-3-yl]methyl}-4-(trifluoromethoxy)benzamide;
N-{[6-(4-bromohophenyl)-5-(2,4-dichlorophenyl)-2-pyrazol-1-yl]methyl)pyridin-3-yl]methyl}-4-(trifluoromethoxy)benzamide;
   in the form of bases or addition salts and also in the form of hydrates or solvates.

As used herein, the following definitions apply:

“Patient” means a warm blooded animal, such as for example rat, mice, dogs, cats, guinea pigs, and primates such as humans.
“Treat” or “treating” means to alleviate symptoms, eliminate the causation of the symptoms either on a temporary or permanent basis, or to prevent or slow the appearance of symptoms of the named disorder or condition.
“Therapeutically effective amount” means a quantity of the compound which is effective in treating the named disorder or condition.
“Pharmaceutically acceptable carrier” is a non-toxic solvent, dispersant, excipient, adjuvant or other material which is mixed with the active ingredient in order to permit the formation of a pharmaceutical composition, i.e., a dosage form capable of administration to the patient. One example of such a carrier is a pharmaceutically acceptable oil typically used for parenteral administration.

In accordance with the invention, the compounds of general formula (I) in which Z represents a N(R₃)XR₄ or N(R₃)COOR₅ may be prepared by the process characterized in that a compound of formula:

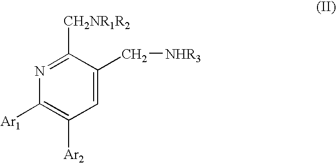

in which the substituents R₁ to R₃ and Ar₁ and Ar₂ are as defined for (I) is treated alternatively:
with an acid of formula R₄CO₂H (III) in which R₄ is as defined for (I), or with an activated derivative of said acid, when it is necessary to prepare a compound of formula (IA) in which X represents a —CO— group; or
with a sulphonyl halide of formula R₄SO₂Hal (IV) in which R₄ is as defined for (I) and Hal represents a halogen atom, preferably chlorine, when it is necessary to prepare a compound of formula (IB) in which X represents an —SO₂— group; or
with an isocyanate of formula R₄—N═C═O (VII) in which R₄ is as defined for (I), to prepare a compound of formula (IC) in which X represents a —CONH— group; or
with an isothiocyanate of formula R₄—N═C═S (VIIa) in which R₄ is as defined above for (I), to prepare a compound of formula (ID) in which X represents a —CSNH— group; or
with an aryloxycarbonyl halide of formula HalCOOR₅ in which R₅ is as defined for a compound of formula (I), when it is necessary to prepare a compound of formula (IE) in which X represents a group N(R₃)COOR₅.

Alternatively, a compound of formula (II) as defined above may be treated with an aryloxycarbonyl halide of formula HalCOOR₅ in which R₅ is as defined for (I), to form an intermediate compound of formula:

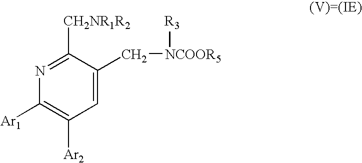

in which the substituents R₁, to R₅ are as defined for (I), which is subsequently treated with an amine of formula R₄R₆NH (VI) in which R₄ and R₆ are as defined for (I), when it is necessary to prepare a compound of formula (IC) in which X represents a group —CON(R₆)—.

According to the invention, the compounds of formula (IF) in which Z represents a group OCONHR₅ are prepared by a process characterized in that a compound of formula:

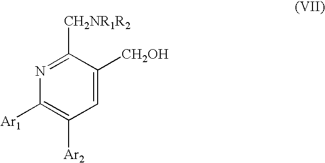

is treated with an isocyanate of formula R₅—N═C═O.

Where appropriate, a compound of formula (I) in which R₃ and/or R₆ represent a (C₁-C₄)alkyl may be prepared by alkylating a compound of formula (I) in which R₃ and/or R₆ is a hydrogen atom, by methods known to the skilled person.

Optionally, the compound of formula (I): (IA), (IB), (IC), (ID), (IE) or (IF) thus obtained is converted into one of its addition salts with an acid.

In the preparation of a compound of formula (IA) in which X represents a —CO— group, it is possible to use an activated derivative of the acid of formula (III), such as an acid chloride or an acid activated by N,N-dicyclohexylcarbodiimide or by benzotriazol-1-yloxytris (dimethylamino)phosphonium hexa fluorophosphate (BOP) benzotriazol-1-yloxytris(pyrrolidino)phosphonium hexafluorophosphate (PyBOP) or 2-(1H-benzotriazol-1-yl)-1,1,3,3-tetramethyluronium tetrafluoroborate (TBTU).

In the preparation of a compound of formula (IB) in which X represents a —SO₂— group, the reaction takes place in the presence of a base such as triethylamine or diisopropylethylamine, in a solvent such as dichloromethane or tetrahydrofuran, at a temperature between the ambient temperature and the reflux temperature of the solvent.

The compounds of formula (IV) are available commercially or are described in the literature, or may be prepared by methods which are described therein, such as in J. Org. Chem. USSR, 1970, 6, 2454-2458; J. Am. Chem. Soc., 1952, 74, 2008; J. Med. Chem., 1977, 20(10), 1235-1239; EP 0 469 984; WO 95/18105.

For example, the compounds of formula (IV) may be prepared by halogenating the corresponding sulphonic acids or their salts, for example their sodium or potassium salts. The reaction takes place in the presence of a halogenating agent such as phosphorus oxychloride, thionyl chloride, phosphorus trichloride, phosphorus tribromide or phosphorus pentachloride, without solvent or in a solvent such as a halogenated hydrocarbon or N,N-dimethyl formamide and at a temperature of between −10° C. and 200° C.

The aryloxycarbonyl halides that are useful in the preparation of a compound of formula (V) are known or are prepared by known methods.

The compounds of formula (II) are prepared in accordance with the reaction scheme below:

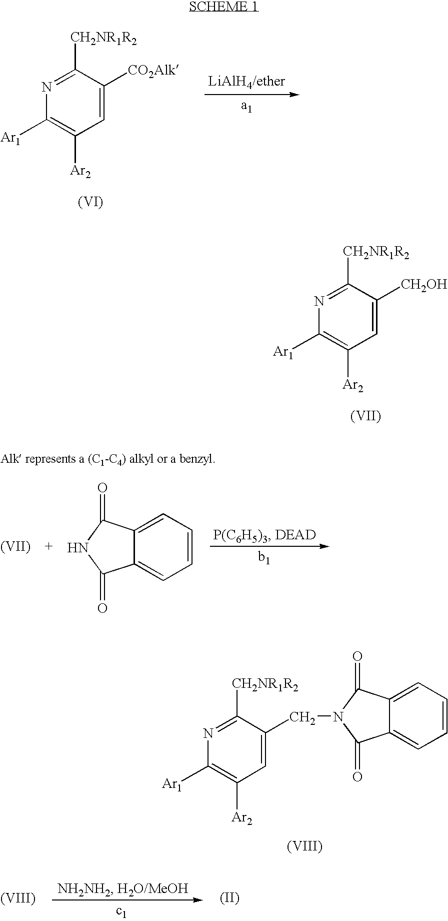

In step a₁ a reducing agent such as LiAlH₄ is used to convert the ester of formula (VI) into alcohol of formula (VII).

In step b₁, the compound of formula (VII) bearing a hydroxymethyl group is employed in a Mitsunobu reaction in the presence of phthalimide to give a compound of formula (VIII), which, when treated with hydrazine hydrate, in the course of a final step c₁, gives the expected compound (II).

The compounds of formula (VI) may be prepared in accordance with the reaction scheme below:

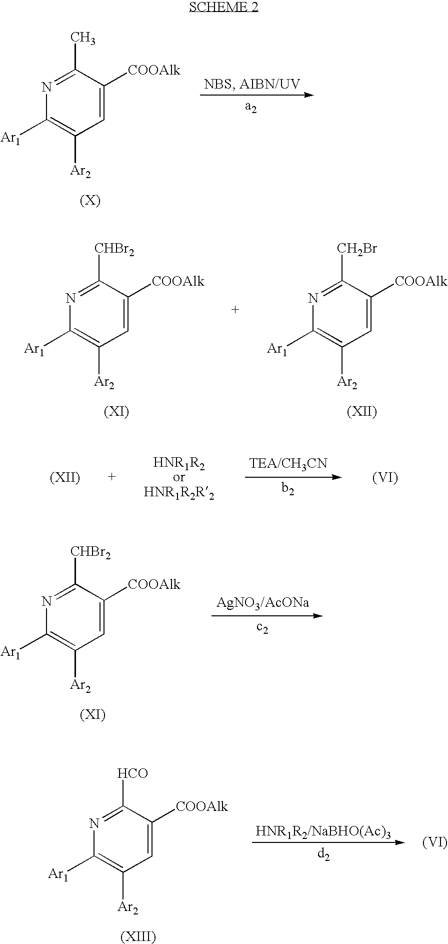

The bromination of step a₃ is carried out by N-bromosuccinimide (NBS) in the presence of AIBN and benzoyl peroxide, under UV irradiation, in a solvent such as CCl₄. This gives a dibromo derivative (XI) and a monobromo derivative (XII).

In step b₂ the monobromo derivative is treated with the amine HNR₁R₂ in the presence of a base such as triethylamine in a solvent such as acetonitrile, to give the compound of formula (VI).

In step c₂ the hydrolysis of the dibromo derivative is carried out with silver nitrate in the presence of sodium acetate, in a solvent such as a water/THF mixture.

In step d₂ an amine of formula HNR₁R₂ is used for treatment in the presence of NaBH(OAc)₃, to give the compound of formula (VI).

The compounds of formula (X) are prepared in accordance with known methods such as those described in WO 03/082191 and WO 2005/00817.

The compounds of formula (VII) may likewise be prepared in accordance with the reaction scheme below:

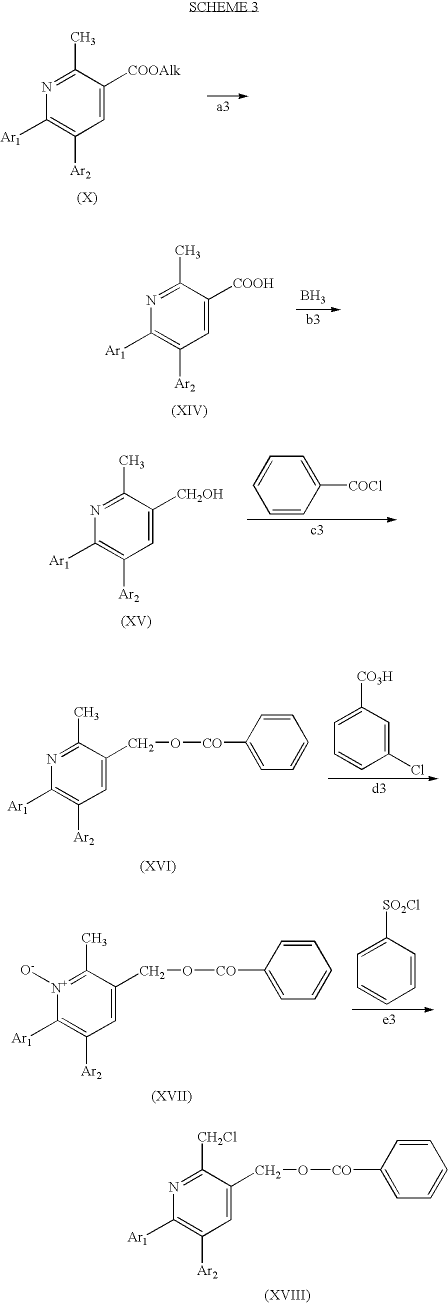

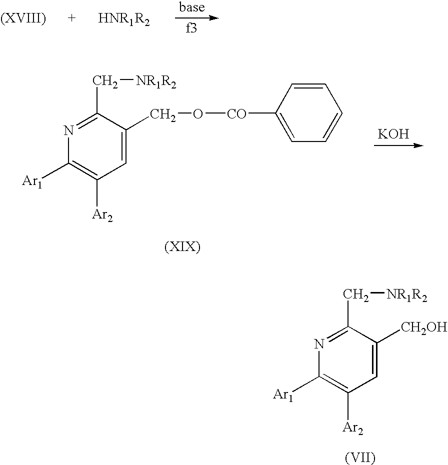

In step a3 the saponification is carried out in a basic medium, for example in the presence of potassium hydroxide. Then step b3 is carried out in the presence of a reducing agent, BH₃ for example.

The esterification with benzoyl chloride (c3) allows the alcohol function to be protected.

In step d3 an oxidizing agent such as meta-chloroperbenzoic acid is used to prepare the pyridine N-oxide derivative of formula (XVII), and then, by a rearrangement (by the method of B. H. Lipshutz et al., Tetrahedron, 1998, 54, 6999-7012), the action of benzenesulphonyl chloride allows the chloro derivative of formula (XVIII) to be prepared, from which it is possible to prepare compounds of formula (VII) variously substituted on the amine function.

The compounds of formula:

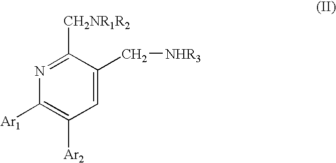

in which:
R₁ to R₃, Ar₁ and Ar₂ are as defined for (I), are new and constitute a further aspect of the present invention.

The compounds of formula:

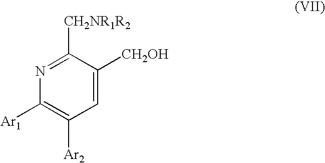

in which:
R₁, R₂, Ar₁ and Ar₂ are as defined for (I), are new and constitute a further aspect of the present invention.

The compounds of formula (VII) are prepared from the compounds of formula (VI) as indicated in scheme 1 or from the compounds of formula (XVIII) as indicated in scheme 3.

According to the present invention, when the group NR₁R₂ is sensitive to reducing agents, it is also possible to prepare a compound of general formula (I) in which Z represents a group N(R₃)R₄ by a process characterized in that:

a) a compound of formula:

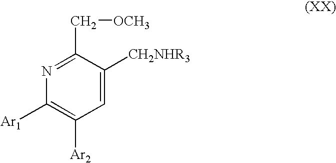

in which the substituents Ar₁, Ar₂ and R₃ are as defined for (I) is treated alternatively:
with an acid of formula R₄CO₂H (III) in which R₄ is as defined for (I), or with an activated derivative of said acid; or
with a sulphonyl halide of formula R₄SO₂Hal (IV) in which R₄ is as defined for (I) and Hal represents a halogen atom, preferably chlorine; or
with an isocyanate of formula R₄—N═C═O (VII) in which R₄ is as defined for (I); or
with an isothiocyanate of formula R₄—N═C═S (VIIa) in which R₄ is as defined above for (I);
   b) the compound thus obtained, of formula:

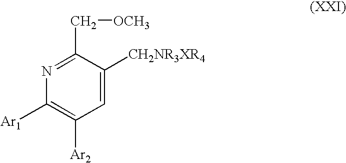

is treated with a dealkylating agent such as BBr₃ or hydrobromic acid; and
c) the compound thus obtained, of formula:

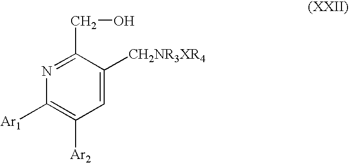

is treated with an amine of formula HNR₁R₂.

Step c) may be carried out by a Mitsunobu reaction, for example, in the presence of diethyl azodicarboxylate and triphenylphosphine.

It is also possible to prepare, as an intermediate, a compound of formula:

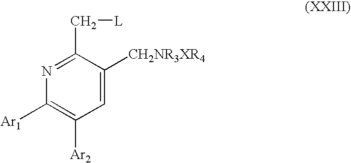

in which L represents a leaving group and subsequently to carry out substitution with an amine HNR₁R₂ by methods which are known to the skilled person, to give the compound of formula (I). This latter process is particularly appropriate for preparing a compound of formula (IA), IB), (IC) or (ID) in which NR₁R₂ represents a tetrazolyl or triazolyl radical.

The compounds of formula (XX) are prepared by the process described in international patent application WO 2006/042955.

The EXAMPLES below describe the preparation of certain compounds in accordance with the invention. These examples are not limitative and serve only to illustrate the present invention.

In the Preparations and in the Examples the following abbreviations are used:
AcOEt: ethyl acetate
AcONa: sodium acetate
AIBN: 2,2′-azobis(2-methylpropionitrile)
AT: ambient temperature
DCM: dichloromethane
DEAD: diethyl azodicarboxylate
pH=2 buffer solution: solution of 16.66 g of KHSO₄ and 32.32 g of K₂SO₄ in 1 litre of water.
DMSO: dimethyl sulphoxide
DIPEA: diisopropylethylamine
DMF: N,N-dimethyl formamide
Et₂O: ether: diethyl ether
2N hydrochloric ether: 2N solution of hydrochloric acid in diethyl ether
iso ether: diisopropyl ether
NaBH(OAc)₃: sodium triacetoxyborohydride
NaHMDS: sodium hexamethylenedisilazane
NBS: N-bromosuccinimide
PTSA: para-toluenesulphonic acid
PyBOP: benzotriazol-1-yloxytris (pyrrolidino)phosphonium hexafluorophosphate
TBTU: 2-(1-benzotriazol-1-yl)yloxytris (pyrrolidino)phosphonium tetrafluoroborate
TEA: triethylamine
THF: tetrahydrofuran

The compounds according to the invention are analysed by LC/UV/MS coupling (liquid chromatography/UT detection/mass spectrometry). Measurements are made of the molecular peak (MH⁺) and the retention time (rt) in minutes (min).

### Conditions A:

The column used is a Symmetry Waters® C18 column sold by Waters, 2.1×30 mm, 3.5 μm, at ambient temperature, flow rate 0.4 ml/minute.

The mobile phase is composed as follows:
solvent A: 0.005% of trifluoroacetic acid (TFA) in water
solvent B: 0.005% of TFA in acetonitrile.

Gradient: the percentage of solvent B varies from 0 to 90% in 10 minutes with a plateau at 90% of B for 5 minutes.

UV detection, is carried out at between 210 nm and 220 nm and mass detection is carried out in positive electrospray ionization (ESI) mode, at atmospheric pressure.

### Conditions B:

The column used is a Symmetry Waters® C18 column sold, by Waters, 2.1×30 mm, 3.5 μm, at ambient temperature, flow rate 0.4 ml/minute.

The mobile phase is composed as follows:
solvent A: 0.005% of trifluoroacetic acid (TFA) in water
solvent B: 0.005% of TFA in acetonitrile.

Gradient: the percentage of solvent B varies from 0 to 90% in 20 minutes with a plateau at 90% of B for 10 minutes.

UV detection is carried out at between 210 nm and 220 nm and mass detection is carried out in positive electrospray ionization (ESI) mode, at atmospheric pressure.

### Conditions C:

The column used is a Symmetry Waters® C18 column sold by Waters, 2.1×30 mm, 3.5 μm, at ambient temperature, flow rate 0.4 ml/minute.

The mobile phase is composed as follows:
solvent A: 10 mM am onium, acetate (pH approximately 7)
solvent B: acetonitrile.

Gradient: the percentage of solvent B varies from 0 to 90% in 10 minutes with a plateau at 90% of B for 5 minutes.

UV detection is carried out at 220 nm and mass detection is carried out in positive electrospray ionization (ESI) mode, at atmospheric pressure.

Unless indicated otherwise, conditions A are the conditions used for LC/MS.

### Preparation 1

### A) Ethyl 6-(4-chlorophenyl)-2-(dibromomethyl)-5-(2,4-dichlorophenyl)nicotinate and ethyl 6-(4-chlorophenyl)-2-(bromomethyl)-5-(2,4-dichlorophenyl)-nicotinate

Ethyl 6-(4-chlorophenyl)-5-(2,4-dichlorophenyl)-2-methyl-nicotinate is placed in solution in 20 ml of CCl₄, and then 4.8 g of NBS, 0.69 g of benzoyl peroxide and 0.35 g of AIBN are added and the mixture is heated at reflux under UV radiation. After a week the reaction mixture is concentrated under vacuum and then the residue is taken up in 200 ml of DCM. The organic phase is washed with 2×200 ml of water, dried over Na₂SO₄, filtered and taken to dryness to give 6.12 g of crude product. The crude product is purified twice on silica, eluting with cyclohexane/AcOEt. Two main fractions are recovered:
1.1 g of the dibromo derivative,
3.42 g of the monobromo derivative.

LC/MS (Conditions B): MH⁺=575.6; rt=21.84 min.

LC/MS (Conditions B): MH⁺=497.8; rt=21.20 min.

### B) 6-(4-Chlorophenyl)-5-(2,4-dichlorophenyl)-2-formyl-nicotinate

6.4 g of the dibromo compound obtained in step A, 7.8 g of sodium acetate and 30.25 g of silver nitrate are placed in 600 ml of THF/water mixture (5/1; v/v). The mixture is heated at reflux for 24 hours. The reaction is treated: the inorganic solid is removed by filtration and then the solvent is evaporated and the crude product is chromatographed on silica, elating with 5% to 12% of AcOEt in cyclohexane in 1 hour. The purified fractions are combined and concentrated to dryness to give, after drying, 1.98 g of the expected compound.

LC/MS: MH⁺=433.8; rt=12.02 min.

### C) Ethyl 6-(4-chlorophenyl)-5-(2,4-dichlorophenyl)-2-(pyrrolidin-1-ylmethyl)nicotinate

1.98 g of the compound obtained in step B, 0.42 ml of pyrrolidine and 1.93 g of NaBH(OAc) are placed in 45.5 ml of DCM and then the mixture is stirred at AT for 2 hours. The mixture is diluted with 50 ml of distilled water and then extracted with 100 ml of DCM; the organic phase is dried over Na₂SO₄, filtered and taken to dryness, to give 2.38 g of the expected compound.

LC/MS: MH⁺=488.9; rt=8.46 min.

### D) Ethyl 6-(4-chlorophenyl)-5-(2,4-dichlorophenyl)-2-(pyrrolidin-2-ylmethyl)nicotinate

3 g of the monobromo compound obtained in step A are placed in 100 ml of acetonitrile. 0.50 ml of pyrrolidine and 0.92 ml of TEA are added. The mixture is stirred at AT for 2 hours. The solvents are evaporated and the residue is taken up in 100 ml of DCM and washed with saturated NaHCO₃ solution. The organic phase is dried over Na₂SO₄, filtered and evaporated to dryness. This gives 2.4 g of the expected compound, which is identical to the product obtained in step C according to TLC analysis (thin-layer chromatography).

### E) 6-(4-Chlorophenyl)-5-(2,4-dichlorophenyl)-2-(pyrrolidin-1-ylmethyl)pyridin-3-ylmethanol

2.38 g of the compound obtained in step C are placed in solution in 20 ml of ether and then at 0° C. 0.27 g of LiAH₄ are added in small spatula-tipfuls. The mixture is left with stirring for 2 hours. The mixture is treated at 0° C. and diluted with 100 ml of ether and then 0.28 ml of distilled water is added, followed by 0.28 ml of 4N NaOH and 0.84 ml of distilled water, until a precipitate is obtained. The mixture is stirred at AT for 1 hour and then the solid formed is filtered off and rinsed with 50 ml of DCM and then 50 ml of MeOH. The solvent is removed by evaporation and the residue is taken up in 100 ml of DCM and the organic phase is washed with 100 ml of distilled water, dried over Na₂SO₄, filtered and taken to dryness, to give 1.92 g of the expected compound.

LC/MS (Conditions B) MH⁺=466.9; rt=11.29 min.

### F) 2-(6-(4-Chlorophenyl)-5-(2,4-dichlorophenyl)-2-(pyrrolidin-1-ylmethyl)pyridin-3-yl)methyl-1H-isoindole-1,3-(2H)dione

1.92 g of the compound obtained in the preceding step, 1.1 g of triphenylphosphine and 0.64 g of phthalimide are placed in 71.5 ml of THF. At −10° C. 0.76 g of DEAD is added dropwise and then the mixture is left at AT overnight. The reaction mixture is diluted with 200 ml of ether. The organic phase is washed with 100 ml of pH=2 buffer and with 100 ml of saturated NaCl solution and is dried over Na₂SO₄, filtered and taken to dryness. This gives 5.85 g of expected product, in crude form. The crude product is purified on silica, eluting with DCM/MeOH from 0 to 3% in 1 hour. The fractions containing the purified product are combined and taken to dryness, to give 510 mg of the expected compound.

LC/MS: MH⁺=575.9; rt=8.56 min.

### G) 1-(6-{4-Chlorophenyl)-5-(2,4-dichlorophenyl)-2-(pyrrolidin-1-ylmethyl}pyridin-3-yl)methanamine

0.51 g of the compound obtained in the preceding step and 0.09 ml of hydrazine monohydrate are placed in solution in 8.84 ml of MeOH and the solution is heated at reflux for 3 hours. The reaction mixture is taken to dryness and then the residue is taken up in 100 ml of distilled water and 100 ml of DCM. The organic phase is washed with 100 ml of saturated NaHCO₃ solution and with 100 ml of saturated NaCl solution, dried over Na₂SO₄, filtered and taken to dryness, to give 423 mg of the expected compound, which is used as it is in the following step.

LC/MS: MH⁺=445.9; rt=6.28 min.

### Preparation 2

### A) 6-(4-Bromophenyl)-5-(2,4-dichlorophenyl)-2-methyl-nicotinic acid

103 g of the ethyl ester of 6-(4-bromophenyl)-5-(2,4-dichlorophenyl)-2-methylnicotinic acid and 67 g of potassium hydroxide are placed in 200 ml of ethanol. After 2 hours with stirring, the mixture is evaporated to dryness and the residue is then washed with Et₂O and extracted with water. The aqueous phase is acidified and the product is extracted with Et₂O and then dried over Na₂SO₄ and filtered, and the solution is taken to dryness. This gives 90 g of the expected acid.

LC/MS: MH⁺=436; rt=10.95 min.

### B) 6-(4-Bromophenyl)-5-(2,4-dichlorophenyl)-2-(methylpyridin-3-yl)methanol

171.5 ml of a 1N solution of BH₃ in THF are diluted in 200 ml of additional THF, and at 0° C. 30 g of the acid prepared in the preceding step, diluted in THF, are added. After the reaction mixture has been stirred at ambient temperature for 12 hours, 100 ml of MeOH are added dropwise at ambient temperature. The mixture is cooled to 0° C., 100 ml of hydrochloric ether are added and then the mixture is left with stirring for 3 hours. It is evaporated to dryness and the residue is washed with saturated NaHCO₃ solution and then extracted with DCM. The extracts are dried over Na₂SO₄ and filtered and the filtrate is taken to dryness, to give 29 g of the expected compound.

LC/MS: MH⁺=422; rt=9.71 min.

### C) 6-(4-Bromophenyl)-5-(2,4-dichlorophenyl)-2-(methylpyridin-3-yl)methyl benzoate

58 g of the compound obtained in the preceding step, 19.6 g of benzoyl chloride and 38.1 ml of triethylamine are placed in 200 ml of DCM and the mixture is left with stirring at ambient temperature for 4 hours. It is washed with saturated NaHCO₃ solution and then extracted with DCM. The extracts are dried over Na₂SO₄ and filtered and the filtrate is taken to dryness, to give 55 g of the expected compound.

LC/MS: MH⁺=526; rt=12.74 min.

### D) (6-(4-Bromophenyl)-5-(2,4-dichlorophenyl)-2-methyl-1-oxidopyridin-3-yl)methyl benzoate

55 g of the compound obtained in the preceding step and 72 g of meta-chloroperbenzoic acid are placed in 200 ml of DCM and the mixture is left with stirring at ambient temperature for 12 hours. The reaction mixture is washed with saturated NaHCO₃ solution and then with water. The aqueous phase is extracted with DCM and the combined organic phases are dried over Na₂SO₄, then filtered and concentrated to dryness to give 55 g of the expected compound.

LC/MS (Conditions C): MH⁺=542; rt=11.09 min.

### E) (6-(4-Bromophenyl)-2-(chloromethyl)-5-(2,4-dichlorophenyl)pyridin-3-yl)methyl benzoate

55 g of the compound obtained in the preceding step, in diluted form, are placed in 100 ml of toluene and the reaction mixture is heated to 80° C. Then 35.8 g of benzenesulphonyl chloride, diluted in 30 ml of toluene, are added over 15 minutes, and the reaction mixture is left with stirring at 80° C. for 72 hours. It is cooled to 0° C. and washed with 5% HCl solution, with saturated Na₂CO₃ solution and then with water. It is extracted with toluene and the extracts are dried over Na₂SO₄ and filtered, and filtrate is taken to dryness, to give 36 g of the expected compound.

LC/MS: MH⁺=560; rt=13.18 min.

### F) (6-(4-Bromophenyl)-5-(2,4-dichlorophenyl)-2-((diethylamino)methyl)pyridin-3-yl)methyl benzoate

6 g of the compound obtained in the preceding step, 2.7 g of diethylamine and 5.9 g of K₂CO₃ are placed in 100 ml of acetonitrile and the mixture is left with stirring for 3 hours at reflux. It is evaporated to dryness and the residue is washed with water and then extracted with DCM. The extracts are dried over Na₂SO₄ and filtered and then the solution is concentrated to dryness, to give 2.7 g of the expected compound.

### Preparation 3

### (6-(4-Bromophenyl)-5-(2,4-dichlorophenyl)-2-((4-methylpiperazin-1-yl)methyl)pyridin-3-yl)methyl benzoate

6 g of the compound obtained in Preparation 2, step 2, and 1 g of N-methylpiperazine are placed in 100 ml of DCM with 1.49 ml of TEA and the mixture is left with stirring at 40° C. for 10 hours. The reaction mixture is washed with water and extracted with DCM. The extracts are dried over Na₂SO₄ and filtered and then the solution is concentrated to dryness, to give 2.5 g of the expected compound.

LC/MS: MH⁺=624; rt=8.56 min.

### Preparation 4

### A) (6-(4-Bromophenyl)-5-(2,4-dichlorophenyl)-2-(1H-pyrazol-1-yl)methyl)pyridin-3-yl)methyl benzoate

1.45 g of pyrazole are placed in 50 ml of THF with 0.85 g of NaH and the reaction mixture is left with stirring at ambient temperature for 2 hours; 6 g of the compound obtained in Preparation 2, step E, are added and the reaction mixture is left with stirring at 70° C. for 3 hours. The reaction mixture is washed with water and extracted with AcOEₜ. The extracts are dried over Na₂SO₄ and filtered and then the solution is concentrated to dryness, to give 3 g of the expected compound.

LC/MS: MH⁺=592; rt=12.49 min.

### B) (6-(4-Bromophenyl)-5-(2,4-dichlorophenyl)-2-(1H-pyrazol-1-ylmethyl)pyridin-3-yl methanol

3 g of the compound obtained in the preceding step and 1.4 g of potassium hydroxide are placed in 100 ml of ethanol and the reaction mixture is left with stirring at ambient temperature for 1 hour. It is evaporated to dryness and the residue is washed with water and extracted with DCM. The extracts are dried over Na₂SO₄ and filtered and then the solution is concentrated to dryness, to give 3 g of the expected compound.

### C) 1-(6-(4-Bromophenyl)-5-(2,4-dichlorophenyl)-2-(1H-pyrazol-1-ylmethyl)pyridin-3-yl)methanamine

This compound is prepared in accordance with the procedure described in steps F and G of Preparation 1.

LC/MS: MH⁺=487; rt=7.54 min.

Compounds of formula (XIX) obtained in Preparations 2 and 3 are used to prepare the compounds of formula (VII) and then the corresponding compounds of formula (II). These compounds are described in Tables I and II below.

Accordingly, the tables which follow illustrate the chemical structures and the physical properties of some intermediates of compounds according to the invention. In these tables Me represents a methyl group.

TABLE I

| | |
|---|---|
| | (VII) |
| 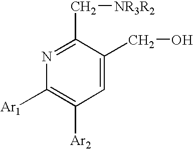 | |

| Inter- | | | | |
|---|---|---|---|---|
| medi- | | | | MH⁺ |
| ate | NR₁R₂ | Ar₁ | Ar₂ | rt (min) |
| 1 | 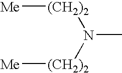 | 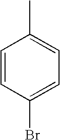 | 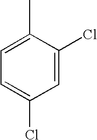 | 521 rt = 7.94 |
| 2 | 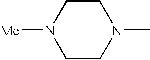 | 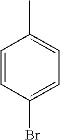 | 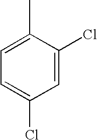 | 520 rt = 7.14 |
| 3 | 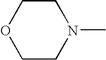 | 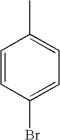 | 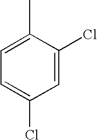 | 463 rt = 7.6 |
| 4 | 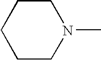 | 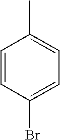 | 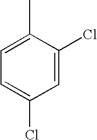 | 461 rt = 7.81 |
| 5 | 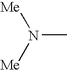 | 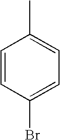 | 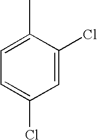 | 421 rt = 7.14 |
| 6 | 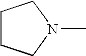 | 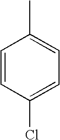 | 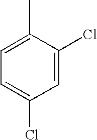 | 446.9 rt = 11.29 Conditions B |

TABLE II

| | |
|---|---|
| | (II) |
| 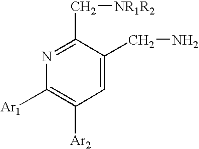 | |

| Inter- | | | | |
|---|---|---|---|---|
| medi- | | | | MH⁺ |
| ate | NR₁R₂ | Ar₁ | Ar₂ | rt (min) |
| 1 | 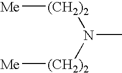 | 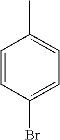 | 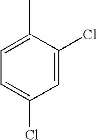 | 520 rt = 6.40 |
| 2 | 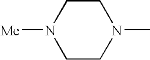 | 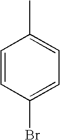 | 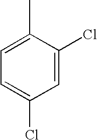 | 519 rt = 5.99 |
| 3 | 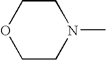 | 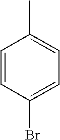 | 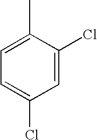 | 462 rt = 6.87 |
| 4 | 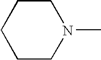 | 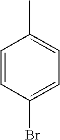 | 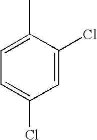 | 460 rt = 6.37 |
| 5 | 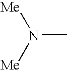 | 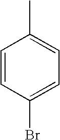 | 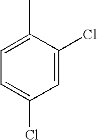 | 460 rt = 6.37 |
| 6 | 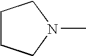 | 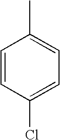 | 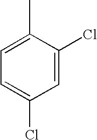 | 445.9 rt = 6.28 |

### Preparation 5

The procedure described in WO 2006/042955 is carried out to prepare:
N-{[6-(4-Bromophenyl)-5-(2,4-dichlorophenyl)-2-(methoxymethyl)pyridin-3-yl]methyl}-4-(trifluoromethyl)-benzamide

### EXAMPLE 1

### Compound 1

### N-{[6-(4-Chlorophenyl)-5-(2,4-dichlorophenyl)-2-(pyrrolidin-1-ylmethyl)pyridin-3-yl]methyl}-4-(trifluoromethoxy)benzamide

The compound obtained in step G of Preparation 1 is placed in solution in DCM and admixed with TEA and then, dropwise, with 0.14 g of 4-trifluoromethoxy-benzenesulphonyl chloride. The mixture is left with stirring at AT for 2 hours. The reaction mixture is diluted with 100 ml of DCM and the organic phase is washed with 100 ml of distilled water, then dried over Na₂SO₄, filtered and taken to dryness, to give 450 mg of crude product. The crude product is purified by chromatography on silica, eluting with DMC/MeOH from 0 to 2% in 1 hour. The fractions containing the purified product are combined and taken to dryness, to give 300 mg of the expected compound in base form. The purified product is converted to the hydrochloride salt in accordance with the standard method. This gives 214 mg of the expected dihydrochloride.

LC/MS: MH⁺=633.9; rt=9.03 min.

### EXAMPLE 2

### Compound 26

### A) N-{[6-(4-Bromophenyl)-5-(2,4-dichlorophenyl)-2-(methoxymethyl)pyridin-3-yl]methyl}-4-(trifluoromethyl)benzamide

This compound is prepared in accordance with WO 2006/042955.

LC/MS: MH⁺=623; rt=12.2 min.

### B) N-{[6-(4-Bromophenyl)-5-(2,4-dichlorophenyl)-2-(hydroxymethyl)pyridin-3-yl]methyl}-4-(trifluoromethyl)benzamide

2 g of the compound from the preceding step are placed in 100 ml of DCM, and then slowly, at −30° C., 12.8 ml of BBr₃ in 1N solution in DCM are added. The mixture is stirred at ambient temperature for 12 hours. It is washed in 200 ml of water and taken up in 100 ml of DCM. The organic phase is dried over Na₂SO₄, filtered and taken to dryness. The crude product obtained is diluted in 200 ml of dioxane/water (50/50; v/v). 1.77 g of K₂CO₃ are added. The mixture is stirred at reflux for 5 hours and evaporated to dryness. The reaction mixture is washed with water and extracted with DCM. The extracts are dried over Na₂SO₄ and filtered and the filtrate is concentrated to dryness. The crude product is purified by chromatography on silica, eluting with DCM/MeOH from 0 to 3% in 1 hour. The purified product is taken to dryness, to give 1.5 g of the expected compound.

LC/MS: MH⁺=609; rt=11.44 min.

### C) N-{[6-(4-Bromophenyl)-5-(2,4-dichlorophenyl)-2-(1H-tetrazol-1-ylmethyl)pyridin-3-yl]methyl}-4-(trifluoromethoxy)benzamide

0.7 g of the compound obtained in the preceding step, 3.73 ml of a 3% solution of tetrazole in acetonitrile and 0.31 g of triphenylphosphine are placed in 100 ml of THF. At 0° C., 0.22 g of DEAD, diluted in 20 ml of THF, is added. The mixture is stirred at 0° C. for 3 hours and then at AT for 2 hours. The reaction mixture is taken to dryness and then washed with water and the residue is taken up in 100 ml of DCM. The organic phase is dried over Na₂SO₄, filtered and taken to dryness. The crude product is purified by chromatography on silica, eluting with DCM/MeOH from 0 to 1% in 1 hour. The purified product is taken to dryness, to give 0.15 g of expected compound.

LC/MS: MH⁺=661; rt=19.1 min.

The table below illustrates the chemical structures and the physical properties of some examples of compounds according to the invention, prepared from the intermediates described above. Within this table, Me represents a methyl group.

The citation of any reference herein should not be construed as an admission that such reference is available as “Prior Art” to the instant application.

The present invention is not to be limited in scope by the specific embodiments describe herein. Indeed, various modifications of the invention in addition to those described herein will become apparent to those skilled in the art from the foregoing description and the accompanying figures. Such modifications are intended to fall within the scope of the appended claims.

Various publications are cited herein, the disclosures of which are incorporated by reference in their entireties.

TABLE III

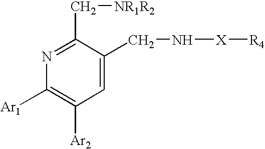

| | | | | | MH⁺ |
|---|---|---|---|---|---|
| Compound | NR₁R₂ | —X—R₄ | Ar₁ | Ar₂ | rt (min) |
| 1 | 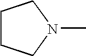 | 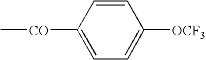 | 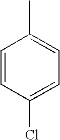 | 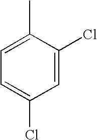 | 633.9 rt = 9.03 |
| 2 | 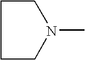 | 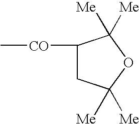 | 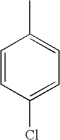 | 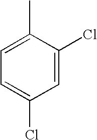 | 602.2 rt = 7.99 |
| 3 | 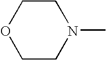 | 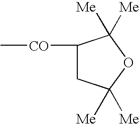 | 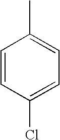 | 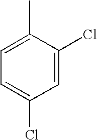 | 616 rt = 8.11 |
| 4 | 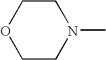 | 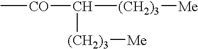 | 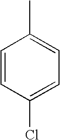 | 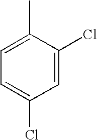 | 616 rt = 9.43 |
| 5 | 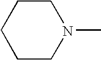 | 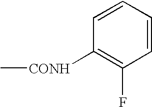 | 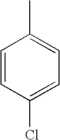 | 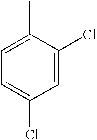 | 597 rt = 8.63 |
| 6 | 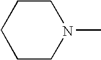 | 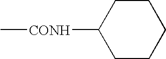 | 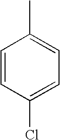 | 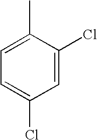 | 585 rt = 10.77 |
| 7 | 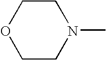 | 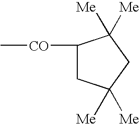 | 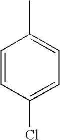 | 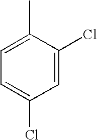 | 614 rt = 9.26 |
| 8 | 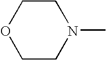 | 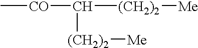 | 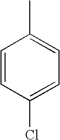 | 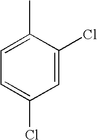 | 588 rt = 8.37 |
| 9 | 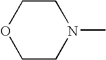 | 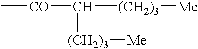 | 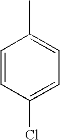 | 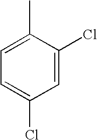 | 602 rt = 13.2 |
| 10 | 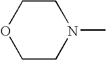 | 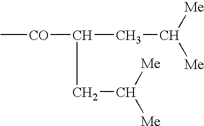 | 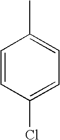 | 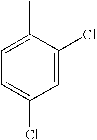 | 616 rt = 9.15 |
| 11 | 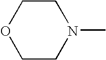 | 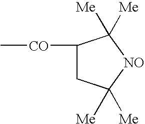 | 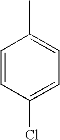 | 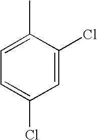 | 630 rt = 10.61 |
| 12 | 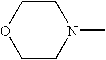 | 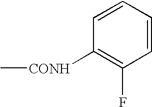 | 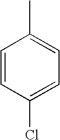 | 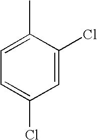 | 599 rt = 8.02 |
| 13 | 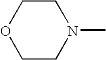 | 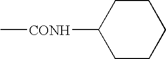 | 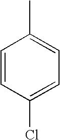 | 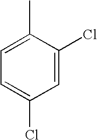 | 587 rt = 8.14 |
| 14 | 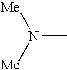 | 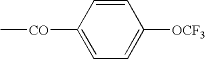 | 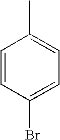 | 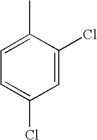 | 608 rt = 8.47 |
| 15 | 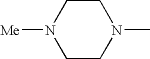 | 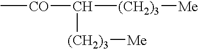 | 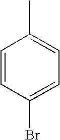 | 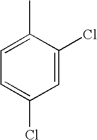 | 673 rt = 8.81 |
| 16 | 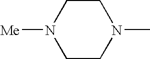 | 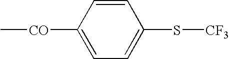 | 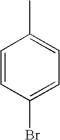 | 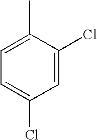 | 723 rt = 8.64 |
| 17 | 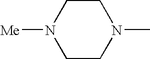 | 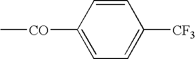 | 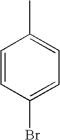 | 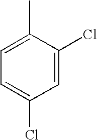 | 691 rt = 8.38 |
| 18 | 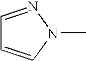 | 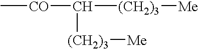 | 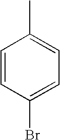 | 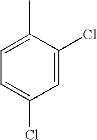 | 641 rt = 12.9 |
| 19 | 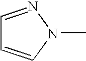 | 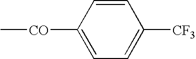 | 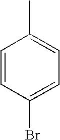 | 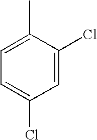 | 659 rt = 12.1 |
| 20 | 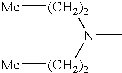 | 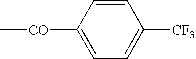 | 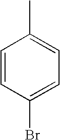 | 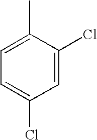 | 692 rt = 9.12 |
| 21 | 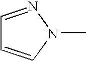 | 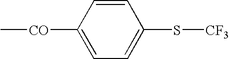 | 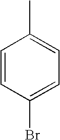 | 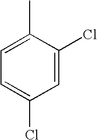 | 691 rt = 12.53 |
| 22 | 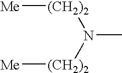 | 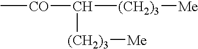 | 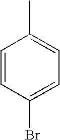 | 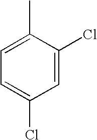 | 674 rt = 9.79 |
| 23 | 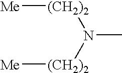 | 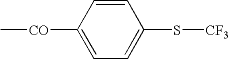 | 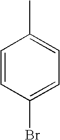 | 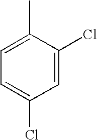 | 724 rt = 9.41 |
| 24 | 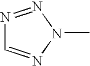 | 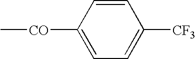 | 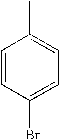 | 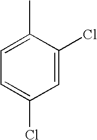 | 661 rt = 19.1 Conditions B |
| 25 | 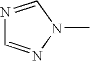 | 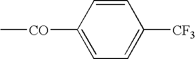 | 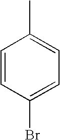 | 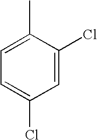 | 660 rt = 17.93 Conditions B |

The compounds of formula (I) possess very good in vitro affinity (IC₅₀≦5×10⁻⁷M) for the cannabinoid CB₁ receptors, under the experimental conditions described by M. Rinaldi-Carmona et al. (FEBS Letters, 1994, 350, 240-244).

The antagonist nature of the compounds of formula (I) was demonstrated by means of the results obtained in the models of inhibition of adenylate cyclase as described in M. Bouaboula et al., J. Biol. Chem., 1995, 270, 13973-13980, M. Rinaldi-Carmona et al., J. Pharmacol. Exp. Ther., 1996, 278, 871-878 and M. Bouaboula et al., J. Biol. Chem., 1997, 272, 22330-22339.

The interaction of a compound according to the invention with the CB1 receptors present in the brain is determined in mice via the ex vivo test of binding of [3H]-CP55940 after an intravenous injection or an oral administration, as described in M. Rinaldi-Carmona et al., FEBS Letters, 1994, 350, 240-244 and M. Rinaldi-Carmona et al., Life Sciences, 1995, 56, 1941-1947, N. Rinaldi-Carmona at al., J. Pharmacol. Exp, Ther., 2004, 310, 905-914 and Rinaldi-Carmona M. et al., JPET 2004, 310, 905-914.

The interaction of a compound according to the invention with the CB1 receptors present in the periphery is determined in mice via the test of reversion of the inhibitory effect of CP55940 on gastrointestinal transit after an oral administration, as described in M. Rinaldi-Carmona et al., JPET, 2004, 310, 905-914.

The toxicity of the compounds of formula (I) is compatible with their use as medicaments.

Thus, according to another of its aspects, the invention provides medicaments for humans or veterinary medicine that comprise a compound of formula (I), or alternatively a solvate or a hydrate of the compound of formula (I).

Thus the compounds according to the invention may be used in the treatment or prevention of diseases involving the cannabinoid CB₁ receptors in humans or animals (particularly in mammals, including, in a non-limiting manner, dogs, cats, horses, cattle and sheep).

For example, and in a non-limiting manner, the compounds of formula (I) are useful as psychotropic medicaments, especially for treating psychiatric disorders including anxiety, depression, mood disorders, insomnia, delirium disorders, obsessive disorders, psychoses in general, schizophrenia, attention deficit and hyperactivity disorders (ADHD) in hyperkinetic children, and also for the treatment of disorders associated with the use of psychotropic substances, especially in the case of a substance abuse and/or dependency on a substance, including alcohol dependency and nicotine dependency.

The compounds of formula (I) according to the invention may be used as medicaments for treating migraine, stress, diseases of psychosomatic origin, panic attacks, epilepsy, motor disorders, in particular dyskinesia or Parkinson's disease, trembling and dystonia.

The compounds of formula (I) according to the invention may also be used as medicaments in the treatment of memory disorders, cognitive disorders, in particular in the treatment of senile dementia and Alzheimer's disease, and also in the treatment of attention or consciousness disorders. Furthermore, the compounds of formula (I) may be useful as neuroprotective agents, in the treatment of ischemia and cranial trauma and the treatment of acute or chronic neurodegenerative diseases, including chorea, Huntington's chorea and Tourette's syndrome.

The compounds of formula (I) according to the invention may be used as medicaments in the treatment of pain: neuropathic pain, acute peripheral pain, chronic pain of inflamatory origin, and pain caused by an anticancer treatment.

The compounds of formula (I) according to the invention may be used as medicaments in human or veterinary medicine, in the treatment of appetite disorders, appetence disorders (for sugars, carbohydrates, drugs, alcohol or any appetizing substance) and/or eating behavioural disorders, especially for the treatment of obesity or bulimia and also for the treatment of type II diabetes or non-insulin-dependent diabetes and for the treatment of dyslipidaemia and metabolic syndrome. Thus the compounds of formula (I) according to the invention are useful in the treatment of obesity and the risks associated with obesity, especially the cardiovascular risks. Furthermore, the compounds of b formula (I) according to the invention may be used as medicaments in the treatment of gastrointestinal disorders, diarrhoea disorders, ulcers, vomiting, bladder and urinary disorders, disorders of endocrine origin, cardiovascular disorders, hypotension, haemorrhagic shock, septic shock, chronic cirrhosis of the liver, hepatic steatosis, steatohepatitis, asthma, Raynaud's syndrome, glaucoma, fertility disorders, interruption of pregnancy, premature birth, inflammatory phenomena, immune system diseases, in particular autoimmune diseases and neuroinflammatory diseases such as rheumatoid arthritis, reactional arthritis, diseases resulting in demyelinization, multiple sclerosis, infectious and viral diseases such as encephalitis, strokes, and also as medicaments for anticancer chemotherapy, for the treatment of Guillain-Barré syndrome and for the treatment of bone diseases and osteoporosis.

According to the present invention, the compounds of formula (I) are most particularly useful for treating psychotic disorders, in particular schizophrenia, attention deficit and hyperactivity disorders (ADHD) in hyperkinetic children; for treating appetite disorders and obesity; for treating memory and cognitive deficits; and for treating alcohol dependency and nicotine dependency, i.e. for withdrawal from alcohol and withdrawal from tobacco.

More particularly, the compounds of formula (I) according to the present invention are useful in the treatment and prevention of appetite disorders, metabolic disorders, gastrointestinal disorders, inflammatory phenomena, immune system diseases, psychotic disorders, alcohol dependency and/or nicotine dependency.

According to one of its aspects, the present invention relates to the use of a compound of formula (I) and solvates or hydrates thereof for treating the disorders and diseases indicated above.

According to another of its aspects, the present invention relates to pharmaceutical compositions comprising, as active principle, a compound according to the invention. These pharmaceutical compositions contain an effective dose of at least one compound according to the invention, a solvate or a hydrate of said compound, and also at least one pharmaceutically acceptable excipient.

Said excipients are chosen according to the pharmaceutical form and the desired mode of administration, from the usual excipients known to those skilled in the art.

The pharmaceutical compositions according to the present invention may contain, along with a compound of formula (I), one (or more than one) other active principle that is useful in the treatment of the disorders and diseases indicated above.

Thus the present invention also provides pharmaceutical compositions containing a compound of formula (I) according to the present invention combined with one (or more than one) active principle chosen from one of the following therapeutic classes:
another cannabinoid CB₁ receptor antagonist or
alloster modulator;
a cannabinoid. CB₂ receptor modulator;
an angiotensin II AT₁ receptor antagonist;
a converting enzyme inhibitor;
a calcium antagonist;
a diuretic;
a beta-blocker;
an antihyperlipaemic or an antihypercholesterolaemic;
an antidiabetic agent;
another anti-obesity agent or agent acting on metabolic disorders;
a nicotine agonist or a partial nicotine agonist;
an antidepressant, an antipsychotic agent or an anxiolytic agent;
an anticancer agent or an antiproliferative agent;
an opioid antagonist;
   and also:
an agent improving memory;
an agent useful in the treatment of alcoholism or withdrawal symptoms;
an agent useful for treating osteoporosis;
a non-steroidal or steroidal anti-inflammatory agent;
an anti-infectious agent;
an analgesic;
an antiasthmatic.

An antidiabetic agent is a compound belonging to one of the following classes: sulphonylureas, biguanidines, alpha-glucosidase inhibitors, thiazolidinedinediones, metiglinides, and also insulin and insulin analogues.

Another anti-obesity agent or agent acting on metabolic disorders is a compound such as a PPAR (Peroxisome Proliferator Activated Receptor) agonist, a dopamine agonist, a leptin receptor agonist, a serotonin reuptake inhibitor, a beta-3 agonist, a CCK-A agonist, an NPY inhibitor, an MC4 receptor agonist, an MCH (Melanin Concentrating Hormone) receptor antagonist, an orexin antagonist, a phosphodiesterase inhibitor, an 11βHSD (11-β-hydroxy steroid dehydrogenase) inhibitor, a DPP-IV (dipeptidyl peptidase IV) inhibitor, a histamine H3 antagonist (or inverse agonist), a CNTF (Ciliary Neurotrophic Factor) derivative, a GHS (Growth Hormone Secretagogue) receptor agonist, a ghrelin modulator, a diacyl glycerol acyl trans f erase (DGAT) inhibitor, a phosphodiesterase (PDE) inhibitor, a thyroid hormone agonist, a glucocorticoid receptor antagonist, a stearoyl-CoA-desaturase (SCD) inhibitor, a phosphate, glucose, fatty acid or dicarboxylate transporter modulator, a 5HT₂ antagonist, a 5HT₆ antagonist or a bombesin agonist.

An agent useful for treating osteoporosis means, for example, bisphosphonates.

According to the present invention, other compounds with antihyperlipaemic, antihypercholesterolaemic, antidiabetic or anti-obesity properties may also be combined. More particularly, compounds belonging to one of the following classes may be combined: PTP 1 B (Protein Tyrosine Phosphase-1B) inhibitors, VPAC 2 receptor agonists, GLK modulators, retinoid modulators, glycogen phosphorylase (HGLPa) inhibitors, glucagon antagonists, glucose-6 phosphate inhibitors, pyruvate dehydrogenase kinase (PKD) activators, RXR, FXR or LXR modulators, SGLT (Sodium-Dependent Glucose Transporter) inhibitors, CETP (Cholesteryl Ester Transfer Protein) inhibitors, squalene synthetase inhibitors, squalene epoxidase inhibitors, triglyceride synthesis inhibitors, LDL (Low-Density Lipoprotein) receptor inducers, IBAT inhibitors, FBPase (fructose-1,6-biphosphatase) inhibitors, CART (Cocaine-Amphetamine-Regulated Transcript) modulators, MC4 (melanocortin 4) modulators, orexin receptor antagonists, and GLP-1 (Glucafon-Like Peptide-1) receptor modulators.

In another aspect of the invention, the compound of formula (I), one of its pharmaceutically acceptable salts or one of their solvates and the other active principle combined may be administered simultaneously, separately or with a time offset.

By simultaneous use is meant the administration of the compounds of the composition according to the invention included in a single pharmaceutical form.

By separate use is meant the administration, at the same time, of the two compounds of the composition according to the invention each included in a separate pharmaceutical form.

By use with a time offset is meant the successive administration of the first compound of the composition of the invention, included in one pharmaceutical form, and then of the second compound of the composition according to the invention, included in a separate pharmaceutical form. In this case, the period of time elapsing between the administration of the first compound of the composition according to the invention and the administration of the second compound of the same composition according to the invention generally does not exceed 24 hours.

In the pharmaceutical compositions of the present invention for oral, sublingual, subcutaneous, intra-muscular, intravenous, topical, local, intratracheal, intranasal, transdermal or rectal administration, the active principle of formula (I) above, or the possible solvate or hydrate thereof, may be administered in a unit form of administration, as a mixture with standard pharmaceutical excipients, to human beings and animals for the prophylaxis or treatment of the above disorders or diseases.

The appropriate unit forms of administration include oral-route forms such as tablets, soft or hard gel capsules, powders, granules and oral solutions or suspensions, sublingual, buccal, intratracheal, intraocular and intranasal administration forms, forms for administration by inhalation, topical, transdermal, subcutaneous, intramuscular or intravenous administration forms, rectal administration forms and implants. For topical application, the compounds according to the invention may be used in creams, gels, ointments or lotions.

By way of example, a unit form of administration of a compound according to the invention in tablet form may comprise the following components:

| | | |
|---|---|---|
| Compound according to the invention: | 50.0 | mg |
| Mannitol: | 223.75 | mg |
| Sodium croscarmellose: | 6.0 | mg |
| Corn starch: | 15.0 | mg |
| Hydroxypropylmethylcellulose: | 2.25 | mg |
| Magnesium stearate: | 3.0 | mg |

Via the oral route, the dose of active principle administered per day may be from 0.01 to 100 mg/kg in one or more dosage intakes, preferentially 0.02 to 50 mg/kg.

There may be particular cases in which higher or lower dosages are appropriate; such dosages are not outside the scope of the invention. According to the usual practice, the dosage that is appropriate to each patient is determined by the doctor according to the mode of administration and the weight and response of said patient.

According to another of its aspects, the present invention also relates to a method of treating the pathologies indicated above which comprises the administration to a patient of an effective dose of a compound according to the invention, or hydrates or solvates.

We claim:

1. A compound of formula (I): 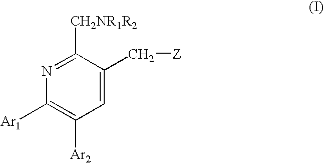
wherein:
Z is N(R₃)XR₄, N(R₃)COOR₅ or OCON(R₃)R₅;
X is —CO—, —SO—, —CON(R₆)— or —CSN(R₆)—;
R₁ and R₂ are, independently of one another, hydrogen or (C₁-C₇)alkyl, or R₁ and R₂, together with the nitrogen atom to which they are bonded: form a saturated or unsaturated 3- to 8-membered heterocyclic radical which may contain one or more other heteroatoms selected from oxygen, sulphur and nitrogen, said heterocyclic radical being optionally substituted by one or more (C₁-C₄)alkyl groups;
R₃ is hydrogen or (C₁-C₄)alkyl;
R₄ is a group selected from the following:
(C₃-C₁₀)alkyl optionally substituted by CF₃;
a nonaromatic (C₃-C₁₂) carbocyclic radical optionally substituted one or more times by identical or different substituents selected from (C₁-C₄)alkyl, hydroxyl, (C₁-C₄)alkoxy, (C₁-C₄)alkylthio and cyano;
a heterocyclic radical of 3 to 8 atoms which contains oxygen, sulphur or nitrogen, is saturated or unsaturated and is optionally substituted by one or more identical or different substituents selected from halogen, (C₁-C₄)alkyl, hydroxyl, trifluoromethyl, (C₁-C₄)alkoxy, trifluoromethoxy, trifluoromethylthio, (C₁-C₄)alkylthio, cyano, nitro and oxo;
indolyl optionally substituted by halogen, (C₁-C₄)alkyl, trifluoromethyl, hydroxyl, (C₁-C₄)alkoxy, trifluoromethoxy, trifluoromethylthio, (C₁-C₄)alkylthio, cyano or nitro;
tetrahydronaphthyl;
naphthyl;
benzothiophenyl;
benzofuryl;
phenyl optionally substituted one or more times by identical or different substituents selected from halogen, (C₁-C₄)alkyl, trifluoromethyl, trifluoromethoxy, hydroxyl, (C₁-C₄)alkoxy, (C₁-C₄)alkylthio, trifluoromethylthio, cyano, nitro, (C₁-C₄)alkanoyl, phenyl, S(O)ₙAlk, OS(O)ₙAlk and NR₇R₈;
benzodioxyl;
phenoxymethylene or 1-phenoxyethylene, the phenyl groups of which are optionally substituted one or more times by identical or different substituents selected from halogen, (C₁-C₄)alkyl, trifluoromethyl, trifluoromethoxy, hydroxyl, (C₁-C₄)alkoxy, (C₁-C₄)alkylthio, trifluoro-methylthio, cyano, nitro, (C₁-C₄)alkanoyl, phenyl, S(O)ₙAlk, OS(O)ₙAlk and NR₇R₈; wherein the methylene or ethylene portion of said phenoxymethylene or 1-phenoxyethylene radical is optionally substituted one or more times by (C₁-C₄)alkyl or (C₃-C₇)cycloalkyl;
phenylcyclopropyl, the phenyl group of which is optionally substituted one or more times by identical or different substituents selected from halogen, (C₁-C₄)alkyl, trifluoromethyl, trifluoromethoxy, hydroxyl, (C₁-C₄)alkoxy, (C₁-C₄)-alkylthio, trifluoromethylthio, cyano, nitro, (C₁-C₄)alkanoyl, phenyl, S(O)ₙAlk, OS(O)ₙAlk and NR₇R₈;
(C₁-C₂)alkylene substituted by one or two identical or different substituents selected from:
(i) a nonaromatic C₃-C₁₂ carbocyclic radical optionally substituted one or more times by (C₁-C₄)alkyl;
(ii) phenyl optionally substituted by one or more identical or different substituents selected from halogen, (C₁-C₄)alkyl, hydroxyl, trifluoromethyl, (C₁-C₄)alkoxy, (C₁-C₄)alkylthio, trifluoromethoxy, trifluoromethylthio, (C₁-C₄)alkanoyl, cyano, nitro, phenyl, S(O)ₙAlk, OS(O)ₙAlk and NR₇R₈; and
(iii) a heterocyclic radical of 3 to 8 atoms which contains oxygen, sulphur or nitrogen, is saturated or unsaturated and is optionally substituted by one or more identical or different substituents selected from halogen, (C₁-C₄)alkyl, hydroxyl, trifluoromethyl, (C₁-C₄)alkoxy, trifluoromethoxy, trifluoromethylthio, (C₁-C₄)alkylthio, cyano and nitro;
additionally, when X is —CON(R₆)— or —CSN(R₆)—, R₄ may be (C₁-C₄)alkanoyl, benzoyl or benzylcarbonyl, the phenyl portion of said benzoyl or benzylcarbonyl radical being optionally substituted by identical or different substituents selected from halogen, (C₁-C₄)alkyl, trifluoromethyl, trifluoromethoxy, hydroxyl, (C₁-C₄)-alkoxy, (C₁-C₄)alkylthio, trifluoromethylthio, cyano, nitro, (C₁-C₄)alkanoyl, phenyl, S(O)ₙAlk, OS(O)ₙAlk and NR₇R₈;
R₅ is phenyl optionally substituted one or more times by identical or different substituents selected from halogen, (C₁-C₄)alkyl, trifluoromethyl, trifluoromethoxy, cyano, nitro, (C₁-C₄)alkoxy, (C₁-C₄)-alkylthio, trifluoromethylthio, S(O)ₙAlk, OS(O)ₙAlk and NR₇R₈;
R₆ is hydrogen or (C₁-C₄)alkyl;
or R₄ and R₆, together with the nitrogen atom to which they are bonded, form a heterocyclic radical of 3 to 8 atoms which may contain a second heteroatom selected from oxygen, sulphur and nitrogen, wherein said heterocyclic radical is optionally substituted one or more times by a (C₁-C₄)alkyl, (C₁-C₄)alkanoyl, NR₇R₈, CONR₇R₈ or phenyl, wherein said phenyl is optionally substituted one or more times by halogen, (C₁-C₄)alkyl, (C₁-C₄)alkoxy, trifluoromethyl, (C₁-C₄)alkylthio, trifluoromethoxy, trifluoromethylthio, OS(O)ₙAlk, S(O)ₙAlk or NR₇R₈;
R₇, and R₈ are, independently of one another, hydrogen or (C₁-C₄)alkyl, or R₇ and R₈, together with the nitrogen atom to which they are bonded, form a saturated heterocyclic radical of 4 to 8 atoms which may contain another heteroatom selected from nitrogen, oxygen and sulphur;
Ar₁ and Ar₂ are, independently of one another, phenyl which is optionally substituted by halogen, (C₁-C₆)alkyl, (C₁-C₆)alkoxy, (C₁-C₆)alkylthio, trifluoromethyl, trifluoromethoxy, trifluoromethylthio, cyano, nitro, S(O)ₙAlk, OS(O)ₙAlk or NR₇R₈;
n is 0, 1 or 2;
Alk is (C₁-C₇)alkyl;
in the form of a base or addition salt, or in the form of a hydrate or solvate.

2. The compound of claim 1 having formula (Ia): 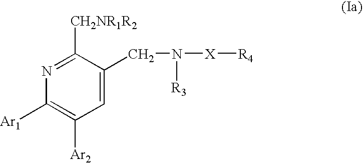
wherein:
X is —CO—, —SO₂— or —CON(R₆)—;
R₁ and R₂ are as defined for (I) in claim 1;
R₃ is hydrogen or (C₁-C₄)alkyl;
R₄ is a group selected from the following:
(C₃-C₁₀)alkyl;
a nonaromatic (C₃-C₁₂) carbocyclic radical, optionally substituted one or more times by (C₁-C₄)alkyl;
a heterocyclic radical of 4 to 8 atoms which contains oxygen, sulphur or nitrogen, is saturated or unsaturated and is optionally substituted by one or more identical or different substituents selected from halogen, (C₁-C₄)alkyl, hydroxyl, trifluoromethyl, (C₁-C₄)alkoxy, trifluoromethoxy, (C₁-C₄)alkylthio, cyano and nitro;
indolyl optionally substituted on the nitrogen atom by halogen, (C₁-C₄)alkyl or (C₁-C₄)alkoxy;
phenyl optionally substituted one or more times by identical or different substituents selected from halogen, (C₁-C₄)alkyl, trifluoromethyl, trifluoromethoxy, (C₁-C₄)alkoxy, (C₁-C₄)alkylthio, trifluoromethylthio, cyano, (C₁-C₄)alkanoyl, phenyl, S(O)ₙAlk and OS(O)ₙAlk; and
benzyl optionally substituted one or more times by identical or different substituents selected from halogen, (C₁-C₄)alkyl, trifluoromethyl, trifluoromethoxy, trifluoromethylthio, (C₁-C₄)alkoxy, cyano, phenyl, S(O)ₙAlk and OS(O)ₙAlk;
R₆ is hydrogen or (C₁-C₄)alkyl;
R₄ and R₆, together with the nitrogen atom to which they are bonded, form a heterocyclic radical of 4 to 8 atoms which may contain a second heteroatom selected from oxygen, sulphur and nitrogen, wherein said heterocyclic radical is optionally substituted one or more times by (C₁-C₄)alkyl, (C₁-C₄)alkanoyl, NR₇R₈, CONR₇R₈ or phenyl optionally substituted one or more times by halogen, (C₁-C₄)alkyl, (C₁-C₄)alkoxy or trifluoromethyl;
R₇ and R₈ are, independently of one another, hydrogen or (C₁-C₄)alkyl, or R₇ and R₈, together with the nitrogen atom to which they are bonded, form a heterocyclic radical selected from piperidinyl, pyrrolidinyl, piperazinyl, N-methylpiperazinyl, azepinyl and morpholinyl;
Ar₁ and Ar₂ are, independently of one another, phenyl optionally
substituted by halogen, (C₁-C₆)alkyl, (C₁-C₆)alkoxy, trifluoromethyl, trifluoromethylthio, trifluoromethoxy, S(O)ₙAlk or OS(O)ₙAlk;
n is 0, 1 or 2;
Alk is (C₁-C₄)alkyl;
in the form of a base or addition salt, or in the form of a hydrate or solvate.

3. The compound of claim 1 having formula (IA): 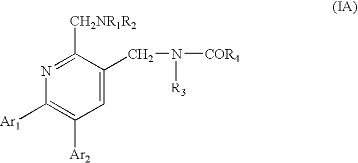
wherein
R₁ and R₂ are, independently of one another, (C₁-C₇)alkyl, or R₁ and R₂, together with the nitrogen atom to which they are bonded, form a radical selected from azeridinyl, azetidinyl, pyrrolidinyl, piperidinyl, azepinyl, piperazinyl, N-methylpiperazinyl, morpholinyl, imidazolyl, pyrazolyl, tetrazolyl and triazolyl;
R₃ is hydrogen or methyl;
R₄ is a group selected from the following:
(C₅-C₁₀)alkyl;
a (C₅-C₇)cycloalkyl optionally substituted one or more times by methyl;
a heterocyclic radical of 4 to 8 atoms which contains oxygen, sulphur or nitrogen, is saturated and is optionally substituted one or more times by methyl; and
phenyl substituted one or more times by groups independently selected from halogen, trifluoromethyl, trifluoromethoxy, trifluoromethylthio, (C₁-C₄)alkoxy, (C₁-C₄)alkylthio, SO₂Alk and OSO₂Alk;
Ar₁ and Ar₂ are, independently of one another, phenyl substituted by one or two substituents independently selected from halogen, methoxy, methylthio, trifluoromethylthio, trifluoromethoxy, SO₂Alk and OSO₂Alk;
in the form of a base or addition salt, or in the form of a hydrate or solvate.

4. The compound of claim 1 having formula (IC): 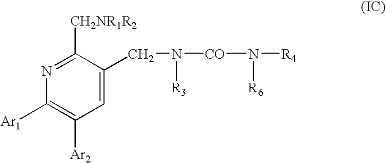
wherein:
R₁ and R₂ are, independently of one another, (C₁-C₇)alkyl, or R₁ and R₂, together with the nitrogen atom to which they are bonded, form a radical selected from azeridinyl, azetidinyl, pyrrolidinyl, piperidinyl, azepinyl, piperazinyl, N-methylpiperazinyl, morpholinyl, imidazolyl, pyrazolyl, tetrazolyl and triazolyl;
R₃ is hydrogen or methyl;
R₄ is a group selected from the following:
(C₅-C₁₀)alkyl;
(C₅-C₇)cycloalkyl optionally substituted one or more times by methyl;
a heterocyclic radical of 4 to 8 atoms which contains oxygen, sulphur or nitrogen, is saturated and is optionally substituted one or more times by methyl; and
phenyl substituted one or more times by groups independently selected from halogen, trifluoromethyl, trifluoromethoxy, trifluoromethylthio, (C₁-C₄)alkoxy, (C₁-C₄)alkylthio, SO₂Alk and OSO₂Alk;
R₆ is hydrogen or methyl;
Ar₁ and Ar₂ are, independently of one another, phenyl substituted by one or two substituents independently selected from halogen, methoxy, methylthio, trifluoromethylthio, trifluoromethoxy, SO₂Alk and OSO₂Alk;
in the form of a base or addition salt, or in the form of a hydrate or solvate.

5. The compound according to claim 3 wherein:
Z is NHCOR₄;
R₁ and R₂ are, independently of one another, (C₁-C₇)alkyl, or R₁ and R₂, together with the nitrogen atom to which they are bonded, form a radical selected from azeridinyl, azetidinyl, pyrrolidinyl, piperidinyl, azepinyl, piperazinyl, N-methylpiperazinyl, morpholinyl, imidazolyl, pyrazolyl, tetrazolyl and triazolyl;
R₄ is 2-propylpentyl, 1-propylbutyl, 5-methylnonyl, 4-methylheptyl, 4-methyl-2,6-dimethylheptyl, cyclopentyl, tetramethylcyclopentyl, cyclohexyl, tetrahydrofuranyl, pyrrolidinyl, 1,1,4,4-tetramethylcyclopentyl, 2,2,5,5-tetramethylfuranyl, 2,2,5,5-tetramethylpyrrolidinyl, or phenyl optionally substituted by halogen, trifluoromethyl, trifluoromethoxy, trifluoromethylthio, SO₂Alk or OSO₂Alk;
Ar₁ and Ar₂ are, independently of one another, phenyl substituted one or more times by substituents independently selected from chlorine, bromine, methoxy and methylthio;
in the form of a base or addition salt, or in the form of a hydrate or solvate.

6. The compound of claim 4 wherein:
Z is —NHCONHR₄;
R₁ and R₂ are, independently of one another, (C₁-C₇)alkyl, or R₁ and R₂, together with the nitrogen atom to which they are bonded, form a radical selected from azeridinyl, azetidinyl, pyrrolidinyl, piperidinyl, azepinyl, piperazinyl, N-methylpiperazinyl, morpholinyl, imidazolyl, pyrazolyl, tetrazolyl and triazolyl;
R₄ is cyclohexyl or phenyl optionally substituted by halogen, methoxy, trifluoromethyl, trifluoromethoxy or trifluoromethylthio;
Ar₁ and Ar₂ are, independently of one another, phenyl optionally substituted one or more times by substituents independently selected from chlorine, bromine, methoxy and methylthio;
in the form of a base or addition salt, or in the form of a hydrate or solvate.

7. A process for preparing a compound of formula (I) wherein Z is N(R₃)XR₄ or N(R₃)COOR₅, characterized in that a compound of formula: 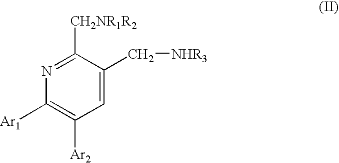
wherein R₁ to R₃ and Ar₁ and Ar₂ are as defined for (I) is treated alternatively:
with an acid of formula R₄CO₂H (III) wherein R₄ is as defined for (I), or with an activated derivative of said acid, when it is necessary to prepare a compound of formula (IA) wherein X is a —CO— group; or
with a sulphonyl halide of formula R₄SO₂Hal (IV) wherein R₄ is as defined for (I) and Hal represents a halogen atom, preferably chlorine, when it is necessary to prepare a compound of formula (IB) wherein X is a —SO₂— group; or
with an isocyanate of formula R₄—N═C═O (VII) wherein R₄ is as defined for (I), to prepare a compound of formula (IC) wherein X is a —CONH— group; or
with an isothiocyanate of formula R₄—N═C═S (VIIa) wherein R₄ is as defined above for (I), to prepare a compound of formula (ID) wherein X is a —CSNH— group; or
with an aryloxycarbonyl halide of formula HalCOOR₅, wherein R₆ is as defined for a compound of formula (I), when it is necessary to prepare a compound of formula (IE) wherein X is a N(R₃)COOR₅ group.

8. A process for preparing a compound of formula (IF) wherein Z is a group OCONHR₅, characterized in that a compound of formula: 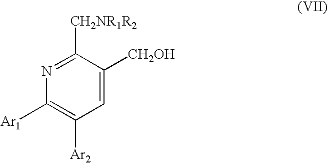
is treated with an isocyanate of formula R₅—N═C═O.

9. A process for preparing a compound of formula (I) wherein Z represents a group N(R₃)XR₄, characterized in that:
a) a compound of formula: 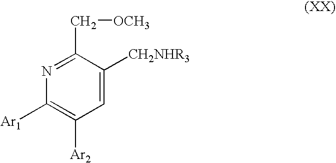
wherein Ar₁, Ar₂ and R₃ are as defined for (I) is treated alternatively:
with an acid of formula R₄CO₂H (III) wherein R₄ is as defined for (I), or with an activated derivative of said acid; or
with a sulphonyl halide of formula R₄SO₂Hal (IV) wherein R₄ is as defined for (I) and Hal is a halogen atom, preferably chlorine; or
with an isocyanate of formula R₄—N═C═O (Vii) wherein R₄ is as defined for (I); or
with an isothiocyanate of formula R₄—N═C═S (VIIa) wherein R₄ is as defined above for (I);
b) the compound thus obtained, of formula: 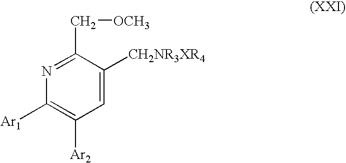
is treated with a dealkylating agent such as BBr₃ or hydrobromic acid; and
c) the compound thus obtained, of formula: 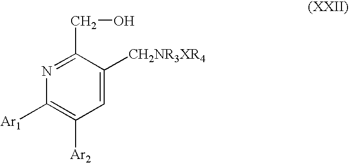
is treated with an amine of formula HNR₁R₂.

10. A compound of formula: 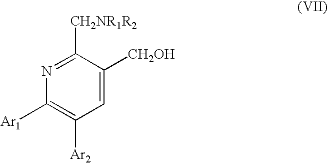
wherein:
R₁, R₂: Ar₁ and Ar₂ are as defined for (I) in claim 1.

11. A compound of formula: 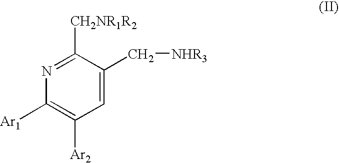
wherein:
R₁to R₃, Ar₁ and Ar₂ are as defined for (I) in claim 1.

12. A medicament characterized in that it comprises a compound of formula (I) according to claim 1 or an addition salt of this compound with a pharmaceutically acceptable acid, or else a hydrate or a solvate of the compound of formula (I).

13. A pharmaceutical composition comprising a therapeutically effective amount of a compound of claim 1 and one or more pharmaceutically acceptable carriers.

14. A method of treating a disease or disorder selected from appetite disorders, metabolic disorders, gastrointestinal disorders, inflammatory phenomena, immune system diseases, psychotic disorders, alcohol dependency and nicotine dependency, comprising administering to a patient in need thereof a therapeutically effective amount of a compound of claim 1.
